(19) United States
(12) Patent Application Publication
(10) Pub. No.: US 2026/0224756 A1
(43) Pub. Date: Aug. 6, 2026

(54) Methods Of Cellular Imaging And Inorganic Nanoimaging Agents For Same

(71) Applicant: Cornell University, Ithaca, NY (US)

(72) Inventors: Ulrich B. Wiesner, Ithaca, NY (US); Karl J. Lewis, Spencer, NY (US); Melia Matthews, Ithaca, NY (US); Nada Naguib, Ithaca, NY (US)

(21) Appl. No.: 19/155,031

(22) PCT Filed: Feb. 9, 2024
(86) PCT No.: PCT/US2024/015289

§ 371 Date: Aug. 8, 2025

Related U.S. Application Data
(60) Provisional application No. 63/444,393, filed on Feb. 9, 2023.
(60) Provisional application No. 63/607,706, filed on Dec. 8, 2023.

(51) Int. Cl. A61K 49/00; G01N 33/58

(52) U.S. Cl. CPC A61K 49/0093; A61K 49/0032; A61K 49/0056; G01N 33/58

(57) ABSTRACT

Methods of imaging of one or more bone(s), one or more musculoskeletal tissue(s), or any portion thereof, or any combination thereof, using one or more inorganic nanoimaging agent(s). Methods may determine a presence, absence, or concentration of an analyte. Compositions may comprise one or more inorganic nanoimaging agent(s). Inorganic nanoimaging agents may comprise one or more dye group(s), one or more targeting group(s), or any combination thereof, and the dye group(s) and/or targeting group(s) may be bonded to a matrix of the inorganic nanoimaging agent. Kits may comprise one or more inorganic nanoimaging agent(s) and/or one or more composition(s) comprising the inorganic nanoimaging agent(s), and may comprise instructions for carrying out a method.

### CROSS REFERENCE TO RELATED APPLICATIONS

This application claims the benefit of U.S. Provisional Application No. 63/444,393, filed on Feb. 9, 2023, and U.S. Provisional Application No. 63/607,706, filed Dec. 8, 2023; the contents of which are hereby fully incorporated herein by reference in their entirety.

### BACKGROUND OF THE DISCLOSURE

Osteocytes, the resident mechanosensor of bone, are responsible for directing the activity of bone resorbing osteoclasts and bone depositing osteoblasts to achieve a functionally healthy skeleton. Their unique 3D environment embedded in bone tissue is important for mechanotransduction in response to tissue level deformation. Importantly, data suggest that changes to the anatomy of osteocytes in vivo and/or changes in the osteocyte microenvironment are responsible for tissue level phenomena seen in osteoporosis. The osteocyte anatomy has been delineated with histological methods, mechanobiology and mechanotransduction mechanisms with cells in vitro and in situ and mechanically driven control of other bone cells using explant studies The next step in understanding osteocytes requires the ability to interrogate their behavior at the cellular/molecular level in real time in vivo, preserving both the native 3D environment as well as paracrine/endocrine interactions.

One target which is critical for proper osteocyte function but has yet to be interrogated with live cell imaging in vivo, are αᵥβ₃ integrins. Early modeling work in osteocyte mechanotransduction identified αᵥβ₃ mediated spot connections between the osteocyte process cell membrane and bone matrix as a key feature of fluid flow mechanical activation of osteocytes. In vitro experiments verified these results by showing that physiologically relevant fluid flow induced calcium (Ca²⁺) signaling activation of MLO-Y4 osteocyte-like cells requires force to be applied at αᵥβ₃ integrin attachments along the process. Moreover, there is evidence that the mechanotransduction complex arrangement changes in disease, implying a dynamic quality to mechanotransducive elements that are likely critical for understanding homeostasis dysregulation. Little is known about integrin dynamics and metabolism in osteocytes, and even less known about their activity in vivo.

Two-photon fluorescence multiphoton microscopy (MPM) uses dual incident beam co-localization to create spot excitation and scans across a single plane to generate an image rather than the bulk cone excitation employed by confocal approaches, decreasing background fluorescence and photobleaching artifacts. Moreover, the longer excitation wavelengths used in MPM allow for increased tissue depth penetration over confocal microscopy. Consequently, MPM provides desirable features for advanced intravital imaging studies.

Traditional small (dye) molecule based fluorescent indicators can be used in vivo but are limited by the lack of spatial specificity and relatively fast bleaching and clearance rates. A class of genetically encoded fluorescent indicators has been established in parallel for the development of intravital MPM techniques that overcomes these limitations. Genetically encoded fluorescent protein constructs typically include genetic material of a green fluorescence protein with tissue, cell, or organelle specific promoter regions to achieve highly targeted expression. They can measure a range of cellular parameters like pH, oxidative stress, cytoskeletal arrangement, and channel specific dynamics. Genetically encoded Ca²⁻ indicators (GECIs) paired with MPM constitute a strong tool for the investigation of cellular level biological events in vivo. While a clear advance over synthetic dyes, their overall brightness and dynamic range are still limited. There is a need to develop alternatives for achieving intravital fluorescent signal with improved optical properties to increase image quality and imaging depth.

Osteocytes are the most numerous cells within bone tissue. They are the resident mechanosensors in bone, translating mechanical stimulation into biochemical signals to control the actions of bone-building osteoblasts and bone-resorbing osteoclasts. Integrin proteins are transmembrane adhesion molecules necessary for osteocyte mechanotransduction. Integrins attach to components of the mineralized bone matrix to form adhesion sites that allow for force amplification of fluid shear stress to initiate cellular mechanosensitive responses.

Other cell types including endothelial cells, fibroblasts, and cancer cells undergoing metastasis also use integrin proteins to sense and respond to their external environment. Spatiotemporal regulation of integrin availability and localization is controlled through endocytosis and recycling. This coordination of integrin dynamics allows for the proteins to be moved to new locations on the cell membrane, which is integral for cell motility, and for signaling from integrin attachments to be modulated. The ability to modulate integrin dynamics impacts cellular response to its environment through adhesion protein localization, number, and availability. There are many different pathways for endocytosis to occur within cells, including ones that are receptor-mediated and one that constitute bulk uptake. Multiple pathways have been implicated in the endocytosis and trafficking of integrins specifically, including clathrin-mediated endocytosis and micropinocytosis.

Endocytosis and trafficking of mechanosensitive transmembrane proteins (i.e. integrins, ion channels, proteoglycans, etc) is a necessary regulatory method for cells to interact with their external environment and to modulate their biochemical response. Dynamic modulation of protein receptor availability controls cellular responsiveness to external stimuli. However no research has been performed on the impact of endocytosis on osteocyte mechanotransduction, which is mediated by transmembrane proteins including integrins, Cav3.2, and P2x7R.

While endocytosis and trafficking of integrins and other mechanosensitive proteins have been thoroughly studied in a number of cell types, osteocytes remain a black box in regards to endocytic pathways and trafficking of mechanosensitive proteins, including integrins. The field of endocytosis and protein trafficking in osteocytes is nascent, and most research that has been done has only been performed in vitro. One genetically modified mouse model with a caveolin-1 knockout found that caveolae and lipid raft domains are critical for normal bone cell and tissue function, which supports the importance of endocytosis in bone. Research has been limited to cell culture and tissue wide effects of genetic knock-out models because visualizing subcellular activities in osteocytes in vivo has proven a difficult task.

### SUMMARY OF THE DISCLOSURE

The present disclosure provides, inter alia, imaging methods. The present disclosure also provides inorganic nanoimaging agents and compositions thereof and uses thereof.

The present disclosure provides imaging methods. A method may be a live-cell method (e.g., a live-cell imaging method, such as, for example, in bone). A method may be an in vivo method, ex vivo method, in vitro method or the like. A method may be a method of imaging of one or more bone(s) (or any portion thereof), one or more musculoskeletal tissue(s) (or any portion thereof), or any combination thereof, using one or more inorganic nanoimaging agent(s). A method may be a method of determining a presence, absence, or concentration of an analyte.

The present disclosure also provides compositions. A composition may comprise one or more inorganic nanoimaging agent(s).

The present disclosure also provides inorganic nanoimaging agents. Inorganic nanoimaging agents may comprise one or more dye group(s), one or more targeting group(s), or any combination thereof, and the dye group(s) and/or targeting group(s) may be bonded to a matrix of the inorganic nanoimaging agent.

The present disclosure also provides kits. A kit may comprise one or more inorganic nanoimaging agent(s) and/or one or more composition(s) comprising the inorganic nanoimaging agent(s). A kit may comprise instructions for use of inorganic nanoimaging agent(s) and/or composition(s) for carrying out a method of the present disclosure.

### BRIEF DESCRIPTION OF THE FIGURES

For a fuller understanding of the nature and objects of the disclosure, reference should be made to the following detailed description taken in conjunction with the accompanying figures.

FIG. 1 shows how C'Dots allow successful intravital imaging of osteocytes in mice after a local injection above the MT3 bone. A—Representative MT3 bone image with MPM excitation at 1090 nm. Green is autofluorescence of bone collagen and red is Cy5 dye within C'Dots. B—Localization of C'Dots within osteocytes. Dual injection of live cell marker Calcein-AM (green) and C'Dots (Red), with overlap shown in yellow. Calcein AM was excited at 920 nm and a 35 μm z-stack was taken. The same z-stack was imaged again at 1090 nm for the C'Dot signal. Channels were merged in FIJI. Signal acquisition for C'Dot fluorescence used a >647 nm longpass filter, and Calcein AM was visualized using a 490-560 nm bandpass filter.

FIG. 2 shows A—The concentration of injected C'Dots influences the subsequent fluorescent intensity in/around osteocytes. Injection volume of 15 μL and incubation time of 2 hours were held constant as the concentration was changed. Data points represent individual cells. N=2 or 3 mice for each group at each concentration. B—RGD and Control C'Dots have distinct trends in mean intensity as incubation time increases. The injection volume of 15 μL and concentration of 10 μM were held constant as the incubation time was changed. Data points represent individual cells. N=1 mouse for each timepoint. Values are normalized to background fluorescence intensity. Error bars±SEM, *=p-value<0.05.

FIG. 3 shows an Experimental Design graphical abstract. Endocytosis disrupting drugs and fluorescent C'Dot nanoparticles were exposed to bone and osteocytes through a local subcutaneous injection above the third metatarsal bone in a mouse hind paw. After a set incubation time, z-stack images of osteocytes in cortical bone were taken at regular intervals with a 2-photon microscope. Images were analyzed for both clearance kinetics of C'Dot signal as well as subcellular localization of signal.

FIG. 4 shows Kinetics of C'Dot clearance can be modulated with pre-incubation of dynamin inhibitor (blue) compared to untreated control (orange). Dynamin inhibition effects C'Dot clearance in opposing ways in male and female mice. Male data was transformed (Y=log(Y+1)) due to near-zero data points prior to ANOVA analysis, untransformed data shown in graph. Stats compare to untreated control (ANOVA, p≤0.05, Error=SEM).

FIG. 5 shows Kinetics of C'Dot clearance can be modulated with pre-incubation of cholesterol inhibitor (pink) compared to untreated control (orange). Male data was transformed (Y=log(Y+1)) due to near-zero data points prior to ANOVA analysis, untransformed data shown in graph. Stats compare to untreated control (ANOVA, p≤0.05, Error=SEM).

FIG. 6 shows Quantification of Subcellular Localization of C'Dots within osteocyte cell bodies. Statistics compare between each experimental state of the different C'Dot groups (Student's T-Test, (*p≤0.05, **p≤0.01). Rim phenotype quantification for individual mice was scored with a by-eye binary of 1=rims, 0=no rims. Violin plots for the results are shown.

FIG. 7 shows Simple Linear Regression lines on C'Dot clearance data for both untreated control and endocytosis inhibited groups (Error=SEM). Data points reaching 0 were removed prior to regression analysis (Male RGD/PEG).

FIG. 8 shows One Phase Decay lines on C'Dot clearance data for both untreated control and endocytosis inhibited groups (Error=SEM). Data points reaching 0 were removed prior to regression analysis (Male RGD/PEG).

FIG. 9 shows RGD C'Dots allow intravital imaging of osteocytes in mice after a local injection above the 3MT bone.

FIG. 10 shows Discrete subcellular localization of RGD C'Dots can be visualized in the cell bodies of osteocytes in vivo. Dynamin inhibition significantly reduces subcell localization in males. (****p≤0.0001).

FIG. 11 shows Kinetics of C'Dot clearance can be modulated with pre-incubation of dynamin inhibitor (blue) or cholesterol inhibitor (pink) compared to untreated control (orange). Dynamin inhibition effects C'Dot clearance in opposing ways in male and female mice. Stats compare to untreated control (ANOVA, p≤0.05, Error=SEM).

FIG. 12 shows an experimental protocol graphic for the injection and imaging of C'Dots in other musculoskeletal tissue types.

FIG. 13 shows a graphical abstract of the injection and imaging of RGD C'Dots in the cortical bone osteocytes of the mouse third metatarsal bone.

FIG. 14 shows example subcellular features that can be observed with C'Dots in cortical bone osteocytes in vivo. The left image depicts osteocyte dendrites and the connections between cells. The images on the right show examples of osteocytes with subcellular localization of signal or diffuse/saturated signal.

FIG. 15 shows a pilot experiment of pH sensing C'Dots locally injected in the same manner as other C'Dots types. They successfully reach the osteocytes and both colors (red and green) used for ratiometric signal detection and pH quantification are visible and overlayed.

FIG. 16 shows a pilot experiment of phalloidin (actin) targeted C'Dots locally injected in the same manner as other C'Dots. They successfully reach osteocytes in murine cortical bone in vivo and strong signal can be seen from osteocyte dendrites, which are known to be high in actin expression.

FIG. 17 shows clearance and intensity of C'Dot signal in osteocytes in vivo along. TAT peptide bonded C'Dots provide an endocytosis control, as they enter the cell through a passive, endocytosis escaping method. The clearance of RGD and PEG C'Dots is also modulated with endocytosis perturbing drugs, Dyngo4a, which inhibits dynamin GTPase activity, and MBCD, which depletes membrane cholesterol. These drugs cause significant changes in uptake and retention of C'Dots into osteocytes.

FIG. 18 shows combined acute and extended C'Dot clearance data from osteocytes in non-drugged and drugged groups in male mice. The acute study is to the left of the vertical black line, the extended study to the right. Stats shown on the graph are from T-tests comparing the final timepoint of the acute study to the first timepoint of the extended study. Colored horizontal line shown the initial uptake of C'Dots compared to the rest of the studies in the same group. Untreated groups in the first column show the expected continuous decay of signal. MBCD has an impact on both C'Dot types, and Dyngo has an impact only on the RGD C'Dots.

FIG. 19 shows combined acute and extended C'Dot clearance data from osteocytes in non-drugged and drugged groups in male mice. The acute study is to the left of the vertical black line, the extended study to the right. Stats shown on the graph are from T-tests comparing the final timepoint of the acute study to the first timepoint of the extended study. Colored horizontal line shown the initial uptake of C'Dots compared to the rest of the studies in the same group. Untreated groups in the first column show the expected continuous decay of signal. MBCD has an impact on both C'Dot types, and Dyngo does not have an impact on either C'Dot type.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, “~,” or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value) or to encompass a genus illustrated by a list of alternatives (e.g., where a list of alternatives is a list of sub-genuses of a genus or species of a genus, and the similarities between the alternatives illustrate the genus), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

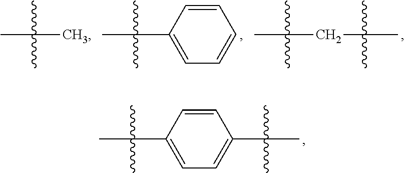

and the like.

The present disclosure provides, inter alia, imaging methods. The present disclosure also provides inorganic nanoimaging agents and compositions thereof and uses thereof.

In an aspect, the present disclosure provides imaging methods. In various examples, a method is an intravital imaging method. In various examples, a method is a live-cell method (e.g., a live-cell imaging method, such as, for example, in bone or the like). In various examples, a method is a theranostic method or the like. In various examples, a method is an in vivo method, ex vivo method, in vitro method or the like. Non-limiting examples of methods are described herein.

In various examples, a method is a method of imaging of one or more bone(s) (or any portion thereof) (such as, for example, one or more cell(s) associated with the bone(s) (e.g., embedded cell(s) or the like) or the like), one or more musculoskeletal tissue(s) (or any portion thereof), or any combination thereof, for example, in a sample or a portion thereof or an individual or a portion thereof, using one or more inorganic nanoimaging agent(s). In various examples a method further comprises targeting, diagnosing, treating, preventing, or any combination thereof, a current or potential disease, disease state, condition, disorder, side effect, or any combination thereof, in the individual.

In various examples, a method comprises contacting a sample, an individual, or any portion thereof with one or more inorganic nanoimaging agent(s). In various examples, a method (e.g., a contacting step of a method) comprises administration (e.g., administration of an effective amount) of one or more inorganic nanoimaging agent(s) (e.g., administration of one or more composition(s) comprising one or more inorganic nanoimaging agent(s)). Various administrations may be used. In various examples, administration is subcutaneous administration, intravenous administration, intraarticular administration, intramarrow administration, or the like, or any combination thereof. In various examples, administration is single dose or multiple doses. In various examples, inorganic nanoimaging agent(s) and/or composition(s) is/are administered proximate to bone(s) (or any portion thereof) (such as, for example, one or more cell(s) associated with the bone(s) (e.g., embedded cell(s) or the like) or the like), musculoskeletal tissue(s) (or any portion thereof), or the like, or any combination thereof of an individual. In various examples, one or more bone(s) (or any portion thereof) (such as, for example, one or more cell(s) associated with the bone(s) (e.g., embedded cell(s) or the like) or the like), one or more musculoskeletal tissue(s) (or any portion thereof), or any combination thereof is put under a mechanical load prior to and/or during imaging. Suitable methods of introducing a mechanical load are known in the art. In various examples, about 1 microliter to about 50 microliters, including all 0.1 microliter values and ranges therebetween) (e.g., of a composition comprising one or more inorganic nanoimaging agent(s)) is administered.

In various examples, a method further comprises an incubation time prior to imaging (e.g., after administration of one or more inorganic nanoimaging agent(s)). In various examples, an incubation time is about 5 minutes to about 24 hours, including all 0.1 minute values and ranges therebetween.

Various bones or the like may be imaged by a method. Non-limiting examples of bones include humerus, tibia, metatarsal, and the like. Various cells or cell types may be imaged by a method. Non-limiting examples of cells or cell types associated with bone (e.g., embedded cells or the like) include osteocytes, osteoblasts, osteoclasts, bone-lining cells, bone marrow cells (e.g., stem cells and the like), blood vessel endothelial cells, and the like, and any combination thereof. Various tissues (e.g., musculoskeletal tissues) may be imaged by a method. Non-limiting examples of tissues include cartilage, marrow, vessels, arteries, skeletal muscle, mineralized bone, bone marrow, vasculature, nerves, tendons, and the like, and any combination thereof. In various examples, tissues comprise tissue cells. Non-limiting examples of tissue cells or tissue cell types include chondrocytes (e.g., cartilage cells), tenocytes (tendon cells), and the like. In various examples, cell(s) or cell type(s) are abnormal cells (e.g., cancerous cell(s) or the like).

In various examples, a method comprises imaging a sample, an individual, or any portion thereof. In various examples, a sample may comprise one or more bone(s), one or more musculoskeletal tissue(s), or one or more cell(s) or cell type(s) (e.g., cell(s) or cell type(s) associated with bone, tissue cell(s) or tissue cell type(s), or the like, or any combination thereof), or the like, or any combination thereof. In various examples, a sample is a biopsy sample or a resected tissue sample. In various examples, a sample is from an individual. In various examples, an individual is a human or non-human animal. Non-limiting examples of non-human animals (which may be mammals) include cows, pigs, mice, rats, rabbits, cats, dogs, and other agricultural animals, pets (such as, for example, dogs, cats, and the like), service animals, and the like.

Various inorganic imaging agents may be used. In various examples, inorganic imaging agent(s) is/are chosen from silica nanoimaging agents, aluminosilicate nanoimaging agents, and the like, and any combination thereof. In various examples, inorganic nanoimaging agent(s) (e.g., at least a portion of, a portion of, or all of the inorganic nanoimaging agent(s)) is/are aluminosilicate nanoimaging agent(s) (e.g., aluminosilicate particles or the like). In various examples, inorganic nanoimaging agent(s) (e.g., at least a portion of, a portion of, or all of the inorganic nanoimaging agent(s)) comprise(s) a peptide group (e.g., a viral peptide group), or the like. In various examples, inorganic nanoimaging agent(s) is/are chosen from nanoparticles, nanorings, nanocages, and the like, and any combination thereof. In various examples, none of the inorganic nanoimaging agent(s) comprises genetic encoding (or is genetically encoded), or the like.

In various examples, an inorganic nanoimaging agent further comprises a plurality of polyethylene glycol (PEG) groups. In various examples, PEG groups are disposed on at least a portion, a portion or all of a surface or surfaces of an inorganic nanoimaging agent (e.g., inorganic nanoimaging agent nanoparticle).

In various examples, inorganic nanoimaging agent(s) comprise a dimension (e.g., a linear dimension or a longest linear dimension) such as, for example, a diameter or the like, of about 1 nm to about 1 μm, including all 0.1 nm values and ranges therebetween (e.g., about 2 nm to 30 nm, or about 2 nm to about 10 nm). In various examples, inorganic nanoimaging agent(s) comprise(s) a longest linear dimension (e.g., a diameter or the like) of about 2 nm to about 1 μm, including all 0.1 nm values and ranges therebetween (such as, for example, about 2 nm to about 30 nm, or about 2 nm to about 10 nm).

International Application No. PCT/US2016/030752 (Title: Ultrasmall Nanoparticles and Methods of Making and Using Same; filed May 4, 2016), International Application No. PCT/US2018/026980 (Title: Sulfur- or Heavy Atom-Containing Nanoparticles, Methods of Making Same, and Uses Thereof; filed Apr. 10, 2018), International Application No. PCT/US2019/026411 (Title: Inorganic Nanocages, and Methods of Making and Using Same; filed Apr. 8, 2019), International Application No. PCT/US2020/028372 (Title: Functionalized Silica Nanorings, Methods of Making Same, and Uses Thereof; filed Apr. 15, 2020), and U.S. patent application Ser. No. 17/501,854 (Title: Super Resolution Optical Microscopy Using Aluminosilicate Nanoparticles; filed Oct. 14, 2021) are each incorporated herein for its disclosure of nanoparticles, nanorings, nanocages, and the like and methods of making same and imaging methods and treatment methods.

In various examples, inorganic nanoimaging agent(s) comprise one or more or all of one or more dye group(s), one or more sulfur atom group(s), one or more radioisotope group(s), one or more heavy atom group(s), one or more iodide group(s), and any combination thereof.

In various examples, inorganic nanoimaging agent(s) (e.g., aluminosilicate nanoimaging agent(s)) comprise(s) at least one dye group, organic dye group, (e.g., dye molecule, organic dye molecule, compound, organic compound, or the like, or any combination thereof), or the like, or any combination thereof. In various examples, a dye group (or an organic dye group) is bonded (e.g., covalently bonded) to a matrix (e.g., aluminosilicate matrix) or network of the individual nanoimaging agent(s) (e.g., aluminosilicate nanoimaging agent(s) (e.g., aluminosilicate nanoparticles)). In various examples, a dye group (e.g., organic dye group) is chosen from fluorescent dye groups, fluorescent protein groups, and the like, and any combination thereof. In various examples, fluorescent dye groups include near infrared (NIR) dye groups and the like. In various examples, organic dye groups are chosen from cyanine dyes, rhodamine dyes (e.g., carborhodamine dyes and the like), coumarin dyes, boron-dipyrromethene (BODIPY) dyes, xanthene dyes, eosin dyes, carbopyronine dyes, methylene blue, fluorescein, Acridine Orange, structural derivatives thereof, and any group derived therefrom, and any combination thereof. In various examples, a dye group is a sensing dye group or a reference dye group. In various examples, a dye group (e.g., a sensing dye group) is capable of sensing pH, sensing redox status, sensing the presence or absence of oxygen, sensing the presence or absence of reactive oxygen species (ROS), sensing the presence or absence of chloride ions, sensing the presence or absence of nitric oxide, sensing the presence or absence of one or more metal(s) and/or metal ion(s), or the like. In various examples, a dye group is chosen from RhG, TMR, Cy3, Cy5, Cy5.5, DY782, and CW800, and any combination thereof.

In various examples, at least a portion of, a portion of, or all of the inorganic nanoimaging agent(s) comprise one or more heavy atom group(s) or the like. In various examples, heavy atom group(s) (e.g., neutral or charged heavy atom(s) or heavy atom group(s)) is/are chosen from iodine atoms, bromine atoms, metal ions (e.g., Au ions, Ag ions, Pb ions, Ti ions, Bi ions, Pt ions, In ions, Sn ions, Sb ions, or Pd ions, or the like), and structural derivatives thereof, and any group derived therefrom, or any combination thereof.

In various examples, at least a portion, a portion, or all of the inorganic nanoimaging agent(s) independently comprise one or more targeting group(s) or the like. In various examples, a targeting group or the like directs an inorganic nanoimaging agent to a specific target in a sample, an individual, or any portion thereof. In various examples, an inorganic nanoimaging agent targets specific cells (or a portion thereof). In various examples, an inorganic nanoimaging agent targets osteocytes, bones marrow cells (e.g., such as, for example, stem cells (hematopoietic stem cells, mesenchymal stem cells, and the like), blood cells (e.g., red blood cells, leukocytes, white blood cells, platelets, and the like, and any combination thereof), lymphocytes, fibroblasts, bone-lining cells, osteoblasts, osteoclasts, chondrocytes, nerve cells, blood vessel cells (e.g., sinusoids, endothelial cells, smooth muscle cells, or the like, or any combination thereof) and/or the like, and/or any combination thereof. Non-limiting examples of targeting group(s) include integrins, transmembrane proteins (e.g., ion channels, connexins, pannexins, purinergic channels and the like), intracellular signaling proteins, cytoskeletal proteins, proteoglycans, and the like, and any combination thereof.

In various examples, at least a portion, a portion, or all of the inorganic nanoimaging agent(s) independently comprise one or more sulfur atom group(s), one or more iodide group(s), one or more drug group(s), one or more radioisotope group(s), or the like, or any combination thereof. Non-limiting examples of radioisotope group(s) include ¹²⁴I, ⁸⁹Zr, ¹¹C, ¹⁵O, ¹⁸F, ⁶⁴Cu, or ⁶⁸Ga, and any combination thereof.

In various examples, electromagnetic radiation is directed into a sample, individual, or any portion thereof. In various examples, electromagnetic radiation is directed into a region (e.g., a region within an individual or sample). In various examples, a region comprises one or more bone(s) (or any portion thereof) (such as, for example, one or more cell(s) associated with the bone(s) (e.g., embedded cell(s) or the like) or the like), one or more musculoskeletal tissue(s) (or any portion thereof), or any combination thereof. In various examples, electromagnetic radiation irradiates one or more inorganic nanoimaging agent(s) (e.g., irradiates and excites one or more dye group(s) of one or more inorganic nanoimaging agent(s)). In various examples, electromagnetic radiation comprises one or more wavelength(s) from about 400 to about 1700 nm, including all 1 nm values and ranges therebetween (e.g., about 400 to about 1200 nm). In various examples, electromagnetic radiation is provided by one or more laser(s) or the like (e.g., directing electromagnetic radiation into a sample, individual, or any portion thereof is carried out using one or more laser(s) or the like (e.g., a single laser or the like)). In various examples, electromagnetic radiation is provided by a tunable wavelength laser or the like.

In various examples, a method produces one or more image(s) (e.g., by one or more imaging process(es)). Various imaging processes may be used. In various examples, one or more image(s) is/are obtained by an intravital fluorescence imaging method, a bioluminescence imaging method, or the like. In various examples, one or more image(s) is/are obtained by multiphoton imaging (e.g., two-photon imaging, three-photon imaging, or the like). In various examples, one or more image(s) is/are obtained by super-resolution imaging (e.g., optical imaging or the like with a resolution below Abbe's diffraction limit). In various examples, one or more image(s) are obtained by optical super-resolution microscopy (OSRM), such as, for example, ground state depletion (GSD) microscopy, stochastic optical reconstruction microscopy (STORM), direct stochastic optical reconstruction microscopy (dSTORM), stimulated emission and depletion (STED), photoactivated localization microscopy (PALM), or the like, or any combination thereof.

Various image(s) (e.g., image(s) of bone(s), cell(s) or cell type(s) associated with bone, musculoskeletal tissue(s), or the like, or any combination thereof) may be obtained by the method. In various examples, an image is obtained by detection of electromagnetic radiation (e.g., electromagnetic radiation emitted by one or more group(s) (e.g., dye group(s)) bonded to an inorganic nanoimaging agent). In various examples, an image is a fluorescence image (e.g., or a sequence of fluorescence images) or the like. In various examples, an image is a super-resolution image (e.g., an image having a resolution below Abbe's diffraction limit. In various examples, a super-resolution image is obtained by processing a sequence of images (e.g., a sequence of fluorescence images). In various examples, at least a portion, a portion, or all of an image comprises (e.g., exhibits) sub-diffraction limit resolution.

In various examples, a method is a method of determining a presence or an absence of an analyte or a concentration of an analyte proximate to or within one or more bone(s) (or any portion thereof) (such as, for example, one or more cell(s) associated with the bone(s) (e.g., embedded cell(s) or the like) or the like), one or more musculoskeletal tissue(s) (or any portion thereof), or any combination thereof, for example, in a sample or in individual in a sample or a portion thereof or an individual or a portion thereof using one or more inorganic nanoimaging agent(s). In various examples a method further comprises targeting, diagnosing, treating, preventing, or any combination thereof, a current or potential disease, disease state, condition, disorder, side effect, or any combination thereof, in the individual.

In various examples, a method of determining a presence, absence, or concentration of an analyte comprises contacting a sample, individual, or any portion thereof with one or more inorganic nanoimaging agent(s); optionally, incubating the sample, individual, or portion thereof for an amount of time; determining a presence, absence, or concentration of an analyte in a first object plane; localizing at least a portion of the inorganic nanoimaging agent(s) in a second object plane; and determining a presence, absence, or concentration of the analyte.

In various examples, one or more of the determining a presence, absence, or concentration of an analyte in a first object plane step, the localizing at least a portion of the inorganic nanoimaging agent(s) in a second object plane step, and the determining a presence, absence, or concentration of the analyte step are carried out with OSRM imaging.

In various examples, determining a presence, absence, or concentration of an analyte in a first object plane comprises ratiometric sensing.

In various examples, localizing at least a portion of the inorganic nanoimaging agent(s) in a second object plane comprises ORSM imaging. In various examples, a second object plane corresponds to at least a portion, a portion, or all of the first object plane.

In various examples, determining a presence, absence, or concentration of the analyte is carried out substantially at or at the position of one or more of the inorganic nanoimaging agent(s). In various examples, determining a presence, absence, or concentration of the analyte uses the presence or the absence or the concentration of the analyte obtained using ratiometric sensing (e.g., in a first object plane) and the localization of the one or more inorganic nanoimaging agent(s) (e.g., obtained using OSRM or the like in a second object plane).

In various examples, a method of determining a presence, absence, or concentration of an analyte further comprises averaging a fluorescence intensity ratio of a desired number of individual inorganic nanoimaging agent(s) in proximity to an individual inorganic nanoimaging agent to assign an average fluorescence intensity ratio to one or more of the individual inorganic nanoimaging agent(s). In various examples, the average fluorescence intensity ratio assigned to the individual inorganic nanoimaging agent(s) corresponds to the presence, absence, or concentration of the analyte in the sample, individual, or the portion thereof.

In various examples, a method of determining a presence, absence, or concentration of an analyte comprises uses one or more inorganic nanoimaging agent(s). In various examples, at least a portion, a portion, or all of the inorganic nanoimaging agent(s) comprises aluminosilicate nanoparticles comprising a plurality of polyethylene glycol (PEG) groups disposed on at least a portion of a surface or all of the surfaces of the inorganic nanoimaging agent(s).

In various examples, a method of determining a presence, absence, or concentration of an analyte comprises imaging. In various examples, a method of determining a presence, absence, or concentration of an analyte comprises OSRM imaging.

A method can investigate various analytes. In various examples, an analyte is chosen from hydrogen ions, oxidants, antioxidants, oxygen, reactive oxygen species (ROS), nitric oxide, chloride ions, metals, metal ions, and the like, and any combination thereof. In various examples, a method determines pH (e.g., an analyte is hydrogen ions and a method determines the concentration of the hydrogen ions). In various examples, a method determines the presence, absence, or concentration of an analyte at (e.g., substantially at) a location of an inorganic nanoimaging agent (e.g., a location of an inorganic nanoimaging agent within a sample, individual, or a portion thereof, or the like).

In various examples, a sensing dye group and a reference dye group are chemically distinct. In various examples, a sensing dye group and a reference dye group comprise different emission wavelengths (e.g., electromagnetic radiation emitted by a sensing dye group comprises a wavelength which is different than a wavelength of electromagnetic radiation emitted by a reference dye group).

A method may have various applications. A method may be used in screening applications or the like. In various examples, a screening method or the like comprises one or more imaging method(s) of the present disclosure. In various examples, a screening method is used to determine the one or more effect(s) of one or more compound(s) (such as, for example, one or more drug(s), one or more drug candidate(s), or the like) (which may be present as a group on a nanoimaging agent or administered independently from the nanoimaging agent) to modulate (e.g., increase, decrease or the like) one or more propert(ies) of a bone (such as for example, bone mass or the like) or the like.

A method may be used to study endocytosis or the like. In various examples, a method of studying endocytosis comprises one or more imaging method(s) of the present disclosure. In various, a method is used to determine one or more effect(s) of one or more compound(s) (such as, for example, one or more drug(s), one or more drug candidate(s), or the like) on endocytosis or the like.

A method may be used to determine (e.g., correlate to a change in) one or more cellular function(s). In various examples, a method (e.g., or one or more image(s) produced by a method) is used to determine a change in one or more cellular function(s), one or more cellular condition(s), or the like, or any combination thereof. Non-limiting examples of cellular functions, conditions, etc. include pH, oxygen concentration, or the like, where any one or all of which are intracellular functions.

A method may method further comprise targeting, diagnosing, treating, preventing, or any combination thereof, a current or potential disease, disease state, condition, disorder, side effect, or any combination thereof, in an individual. In various examples, a current or potential disease, disease state, condition, disorder, side effect, or the like, or any combination thereof, is chosen from infections, cancers, inflammatory conditions/diseases, and the like, and any combination thereof. Non-limiting examples of current or potential diseases, disease states, conditions, disorders, and side effects include marrow metabolism, stem cell dysfunction, sickle cell disease, osteoporosis disease, cellular mechanotransduction, osteoporosis, osteoarthritis, tendonitis, and the like, and any combination thereof.

In various examples, a current or potential disease, disease state, condition, disorder, or side effect is cancer. Non-limiting examples of cancer include bone cancer, leukemia, and the like, and any combination thereof. In various examples, (e.g., where a current or potential disease is cancer) a method further comprises one or more chemotherapy treatment(s), one or more radiation treatment(s), one or more photodynamic therapy treatment(s), one or more surgical intervention(s), or the like, or any combination thereof. These individual treatment(s) (e.g., the one or more additional step(s)) may be carried out before and/or after and/or during obtaining the image(s). In various examples, a method further comprises administering one or more compound(s) (e.g., one or more drug(s) or the like). Individual treatment(s) (e.g., the one or more additional step(s)) may be carried out before and/or after and/or during a method (e.g., before and/or after and/or during obtaining one or more image(s)).

In an aspect, the present disclosure provides compositions. In various examples, a composition comprises one or more inorganic nanoimaging agent(s) of the present disclosure. Non-limiting examples of compositions are described herein.

In various examples, a composition comprises one or more inorganic nanoimaging agent(s) of the present disclosure.

In various examples, a composition is suitable for administration to an individual. In various examples, a composition is suitable for subcutaneous administration, intravenous administration, intraarticular administration, intramarrow administration, or the like to an individual.

Compositions can comprise various concentrations (e.g., concentrations of inorganic nanoimaging agents). In various examples, a composition comprises inorganic nanoimaging agent(s) at a concentration of about 10 nanomolar to about 100 micromolar, including all 0.1 micromolar values and ranges therebetween (e.g., about 1 micromolar to about 5 micromolar or about 5 micromolar to about 100 micromolar).

In an aspect, the present disclosure provides inorganic nanoimaging agents. In various examples, an inorganic nanoimaging agent is an inorganic nanoimaging agent of the present disclosure. In various examples, an inorganic nanoimaging agent comprises one or more targeting group(s) independently bonded to a matrix of the inorganic nanoimaging agent and/or one or more dye group(s) independently bonded to the matrix of the inorganic nanoimaging agent. In various examples, a matrix comprises a silicate or aluminosilicate matrix. In various examples, an inorganic nanoimaging agent further comprises a plurality of polyethylene glycol (PEG) groups disposed on at least a portion of a surface or all of the surfaces of the inorganic nanoimaging agent(s). In various examples, an inorganic nanoimaging agent comprises a diameter of about 1 nm to about 8 nm. In various examples, an inorganic nanoimaging agent comprises a diameter of about 7 nm or less. In various examples, one or more dye group(s) are independently chosen from RhG, TMR, Cy3, Cy5, Cy5.5, DY782, and CW800. In various examples, one or more targeting group(s) are independently chosen from integrin binding peptide(s), TAT viral peptide(s), and phalloidin targeting group(s).

In an aspect, the present disclosure provides kits. In various examples, a kit comprises one or more inorganic nanoimaging agent(s) and/or one or more composition(s) comprising the inorganic nanoimaging agent(s), and instructions for use of the inorganic nanoimaging agent(s) and/or the composition(s) for carrying out a method of the present disclosure. Non-limiting examples of compositions are described herein. In various examples, a kit comprises a composition of the present disclosure. In various examples, a kit comprises one or more inorganic nanoimaging agent(s) of the present disclosure.

In various examples, a kit comprises inorganic nanoimaging agent(s) individually comprising at least one dimension (which may be a longest linear dimension, such as, for example, a diameter) of about 1 to about 30 nm, including all 0.1 nm values and ranges therebetween (e.g., about 2 nm to about 10 nm).

In an aspect, the present disclosure provides treatment methods. In various examples, a treatment method comprises administration of a compound (such as, for example, a drug, a drug candidate, or the like) (which may be in the absence of administration of a nanoimaging agent) described in the present disclosure to an individual (as described herein), where the individual is treated (e.g., as described herein).

The following Statements describe various examples of inorganic nanoimaging agents, compositions, and methods of the present disclosure that are not intending to be limiting in any manner:

Statement 1. A method of imaging of one or more bone(s) (or any portion thereof) (such as, for example, one or more cell(s) associated with the bone(s) (e.g., embedded cell(s) or the like) or the like), one or more musculoskeletal tissue(s) (or any portion thereof), or any combination thereof in a sample or in individual in a sample or a portion thereof or an individual or a portion thereof, the method comprising contacting the sample or the individual with a plurality of one or more inorganic nanoimaging agent(s) (such as, for example, contacting the sample or the individual with one or more composition(s), each composition comprising a plurality of one or more inorganic nanoimaging agent(s)), where each inorganic nanoimaging agent(s) of the plurality of inorganic nanoimaging agent(s) independently comprises one or more dye group(s), one or more sulfur atom group(s), one or more radioisotope group(s), one or more heavy atom group(s), or the like, or any combination thereof, or any combination thereof, where the dye group(s), the sulfur atom group(s), the radioisotope group(s), the heavy atom group(s), or the like, if present, are independently disposed on a surface of an inorganic nanoimaging agent (e.g., covalently or non-covalently bonded to a surface of an inorganic nanoimaging agent or the like) covalently or non-covalently bonded (e.g., chelated or the like) to the matrix (e.g., silica matrix, aluminosilicate matrix, or the like) of an inorganic nanoimaging agent; directing excitation electromagnetic radiation into the sample or the individual, thereby exciting at least one of the one or more dye molecule(s), if present, one or more radioisotope(s), if present, or one or more iodide(s), if present, or the combination thereof resulting in emission (or excitation and emission) of electromagnetic radiation from the dye molecule(s), if present, the radioisotope(s), if present, or the iodide(s), if present, or the combination thereof; detecting at least a portion of the emitted electromagnetic radiation, the detected electromagnetic radiation having been emitted by the dye molecule(s), if present, the radioisotope(s), if present, or the iodide(s), if present, or the combination thereof; and processing signals corresponding to the detected electromagnetic radiation to provide one or more image(s) of the bone(s) (or the portion thereof), the musculoskeletal tissue(s) (or the portion thereof), or the combination thereof in the sample or the individual.
Statement 2. A method according to Statement 1, where the inorganic nanoimaging agent(s) is/are chosen from silica nanoimaging agents, aluminosilicate nanoimaging agents, or the like, or any combination thereof.
Statement 3. A method according to Statement 1 or 2, where the inorganic nanoimaging agent(s) is/are chosen from nanoparticles, nanorings, nanocages, or the like, or any combination thereof.
Statement 4. A method according to any one of the preceding Statements, where the inorganic nanoimaging agent(s) comprise a longest linear dimension (such as, for example, a diameter or the like) of about 2 nanometers (nm) to about 1 micrometer (micron), including all 0.1 nm values and ranges therebetween (about 2 nm to about 10 nm).
Statement 5. A method according to any one of the preceding Statements, where at least a portion or all of the dye groups(s) is/are organic dye group(s) or a combination of organic dye group(s).
Statement 6. A method according to any one of the preceding Statements, where at least a portion or all of the dye group(s) (e.g., organic dyes or the like) is/are fluorescent dye groups(s), fluorescent protein groups(s), or the like, or any combination thereof.
Statement 7. A method according to Statement 5 or 6, where the organic dye group(s) is/are chosen from cyanine dyes, rhodamine dyes (e.g., carborhodamine dyes and the like), coumarin dyes, boron-dipyrromethene (BODIPY) dyes, xanthene dyes, eosin dyes, carbopyronine dyes, methylene blue, fluorescein, Acridine Orange, structural derivatives thereof, or any group derived therefrom, or any combination thereof, and/or the heavy atom group(s) (which may be neutral or charged heavy atoms/heavy atom groups) is/are chosen from iodine atom, bromine atom, metal ions (such as, for example, Au ions, Ag ions, Pb ions, Ti ions, Bi ions, Pt ions, In ions, Sn ions, Sb ions or Pd ions, and the like), and structural derivatives thereof, and any group derived therefrom, or any combination thereof.
Statement 8. A method according to any one of the preceding Statements, where the contacting is administering one or more composition(s) of the present disclosure (such as, for example, composition(s) of Statements 34 or 35 or the like) (such as, for example, an effective amount of the composition) to the individual.
Statement 9. A method according to any one of the preceding Statements, where the electromagnetic radiation is directed into the individual.
Statement 10. A method according to Statement 9, where the electromagnetic radiation is directed into a region, where the region is within the individual.
Statement 11. A method according to any one of the preceding Statements, where the electromagnetic radiation comprises one or more wavelengths from about 400 to about 1700 nm, including all nm values and ranges therebetween (e.g., about 400 to about 1200 nm).
Statement 12. A method according to any one of the preceding Statements, where the irradiation is carried out using one or more laser(s) (e.g., a single laser or the like) or the like.
Statement 13. A method according to any one of the preceding Statements, where the electromagnetic radiation is provided by tunable wavelength laser or the like.
Statement 14. A method according to any one of the preceding Statements, where the directing, detecting, and processing is fluorescence imaging, bioluminescence imaging, or the like, or any combination thereof.
Statement 15. A method according to any one of the preceding Statements, where at least a portion or all of the inorganic nanoimaging agent(s) is/are aluminosilicate nanoimaging agent(s) (e.g., aluminosilicate nanoparticles or the like) (e.g., aluminosilicate nanoparticle(s) comprising at least one organic dye molecule covalently bonded to the aluminosilicate network of the individual aluminosilicate nanoparticles) and the imaging is super-resolution imaging (e.g., optical imaging or the like with resolution below Abbe's diffraction limit).
Statement 16. A method according to Statement 15, where the image(s) are fluorescence image(s) (which independently may be a super resolution image) or a sequence of fluorescence image(s) which can be processed to obtain a super-resolution optical image (e.g., an optical image or the like with resolution below Abbe's diffraction limit) of the sample or portion thereof or the individual or a portion thereof.
Statement 17. A method according to any one of the preceding Statements, where the method is an optical super-resolution microcopy method including, but not limited to, ground state depletion (GSD) microscopy, stochastic optical reconstruction microscopy (STORM), direct stochastic optical reconstruction microscopy (dSTORM), stimulated emission and depletion (STED), or photoactivated localization microscopy (PALM).
Statement 18. A method according to any one of the preceding Statements, the method further comprising targeting, diagnosing, treating, preventing, or any combination thereof, a current or potential disease, disease state, condition, disorder, side effect, or any combination thereof, in an individual.
Statement 19. A method according to Statement 18, where the sample is a biopsy sample or a resected tissue sample.
Statement 20. A method according to Statement 18 or 19, where the current or potential disease, disease state, condition, disorder, side effect, or any combination thereof, is chosen from infections, cancers, inflammatory conditions/diseases, and any combination thereof.
Statement 21. A method according to any one of Statements 18-20, where at least a portion or all of the inorganic nanoimaging agent(s) independently comprises one or more drug group(s), one or more radioisotope group(s), or the like, or any combination thereof, and the contacting results in treatment of the individual.
Statement 22. A method according to any one of the preceding Statements, where at least a portion or all of the inorganic nanoimaging agent(s) independently comprises one or more targeting group(s) or the like.
Statement 23. A method according to any one of Statements 18-22, where the current or potential disease is cancer, and the method further comprises one or more chemotherapy treatment(s), one or more radiation treatment(s), one or more photodynamic therapy treatment(s), one or more surgical intervention(s) (e.g., surgical procedure(s), or the like), or the like, or any combination thereof.
Statement 24. A method of determining a presence or an absence of an analyte or a concentration of an analyte proximate to or within one or more bone(s) (or any portion thereof) (such as, for example, one or more cell(s) associated with the bone(s) (e.g., embedded cell(s) or the like) or the like), one or more musculoskeletal tissue(s) (or any portion thereof), or any combination thereof in a sample or in individual in a sample or a portion thereof or an individual or a portion thereof using one or more inorganic nanoimaging agent(s) comprising contacting the sample or individual with one or more (and/or a plurality of) inorganic nanoimaging agent(s) (as described herein, such as, for example, silica, aluminosilicate nanoparticles, or the like, or any combination thereof) each inorganic nanoimaging agent comprising one or more reference dye groups(s), where each reference dye group is covalently bound to and encapsulated in the network of the inorganic nanoimaging agent, and/or one or more sensing dye groups(s), where the sensing groups are capable of interacting with the sample or the portion thereof or the individual or the portion thereof, and a plurality of polyethylene glycol (PEG) groups disposed on at least a portion of a surface or all of the surfaces of the inorganic nanoimaging agent; optionally, incubating the inorganic nanoimaging agent(s) with the sample or in the individual; determining a presence or an absence or a concentration of the analyte in an individual region of a first object plane using ratiometric sensing; localizing with resolution below Abbe's diffraction limit at least a portion of or all of the individual inorganic nanoimaging agent(s) (e.g., in a second object plane, where the second object plane corresponds to at least a portion or all of the first object plane, using, for example, optical super-resolution microscopy (OSRM) imaging or the like); determining a presence or an absence of the analyte or the concentration of the analyte substantially at or at the position of one or more of the inorganic nanoimaging agent(s) using the presence or the absence or the concentration of the analyte obtained using the ratiometric sensing and the localization of the inorganic nanoimaging agent(s) (e.g., obtained using OSRM or the like); and optionally, averaging the fluorescence intensity ratio of a desired number of individual inorganic nanoimaging agent(s) in proximity to an individual inorganic nanoimaging agent to assign an average fluorescence intensity ratio to one or more or the individual inorganic nanoimaging agent(s), where the average fluorescence intensity ratio assigned to the individual inorganic nanoimaging agent(s) corresponds to the presence or the absence of the analyte or the concentration of the analyte in the sample or the portion thereof or the individual or the portion thereof.
Statement 25. A method according to Statement 24, where the determining the presence or the absence or the local concentration of the analyte in the individual region of a detecting plane using ratiometric sensing and the localizing with resolution below Abbe's diffraction limit at least a portion of or all of the individual inorganic nanoimaging agent(s) in the second object plane are each carried out using OSRM imaging.
Statement 26. A method according to Statement 24 or 25, where the presence or the absence of the analyte or the concentration of the analyte in the sample or the portion thereof or the individual or the portion thereof is determined substantially at one or more of the inorganic nanoimaging agent(s).
Statement 27. A method according to any one of Statements 24-26, where the analyte is chosen from hydrogen ions, oxidants, antioxidants, oxygen, reactive oxygen species (ROS), nitric oxide, chloride ions, metals, metal ions, and the like, and any combination thereof.
Statement 28. A method according to any one of Statements 24-27, where the analyte is hydrogen ions and the local pH substantially at or at the position of at least a portion or all of the inorganic nanoimaging agent(s) in the sample or the portion thereof or the individual or the portion thereof is determined.
Statement 29. A method according to any one of Statements 24-28, where the individual sensing dye group(s) is/are capable of sensing pH, sensing redox status, sensing the presence or absence of oxygen, sensing the presence or absence of reactive oxygen species (ROS), sensing the presence or absence of chloride ions, sensing the presence or absence of nitric oxide, sensing the presence or absence of one or more metal(s) and/or metal ion(s), or the like.
Statement 30. A method according to any one of Statements 24-29, where at least a portion or all of the inorganic nanoimaging agent(s) (e.g., silica nanoparticles, aluminosilicate nanoparticles, or the like) further comprise one or more targeting group(s), one or more therapeutic group(s), one or more diagnostic group(s), or any combination thereof.
Statement 31. A method according to any one of Statements 24-30, where the method comprises an OSRM method chosen from ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM).
Statement 32. A method according to any one of Statements 24-30, where the inorganic nanoimaging agent(s) individually have at least one dimension (which may be a longest linear dimension, such as, for example, a diameter) of about 1 to about 30 nm, including all 0.1 nm values and ranges therebetween.
Statement 33. A method according to any one of Statements 24-32, where the individual is a human or a non-human animal (e.g., mammal or the like).
Statement 34. A composition (e.g., for imaging one or more bone(s) (or any portion thereof) (such as, for example, one or more cell(s) associated with the bone(s) (e.g., embedded cell(s) or the like) or the like), one or more musculoskeletal tissue(s) (or any portion thereof), or any combination thereof in a sample or in individual comprising a plurality of one or more inorganic nanoimaging agent(s), where each inorganic nanoimaging agent(s) of the plurality of inorganic nanoimaging agent(s) independently comprises one or more dye group(s), one or more sulfur atom group(s), one or more radioisotope group(s), one or more heavy atom group(s), or the like, or any combination thereof, or any combination thereof, where the dye group(s), the sulfur atom group(s), the radioisotope group(s), the heavy atom group(s), targeting group(s), or the like, if present, are independently disposed on a surface of an inorganic nanoimaging agent (e.g., covalently or non-covalently bonded to a surface of an inorganic nanoimaging agent or the like) covalently or non-covalently bonded (e.g., chelated or the like) to the matrix (e.g., silica matrix, aluminosilicate matrix, or the like) of an inorganic nanoimaging agent (e.g., as described herein or the like).
Statement 35. A composition according to Statement 34, where at least a portion or all of the inorganic nanoimaging agent(s) independently comprises one or more drug group(s), one or more radioisotope group(s), or the like, or any combination thereof.
Statement 36. A kit comprising one or more inorganic nanoimaging agent(s) and/or one or more composition(s) comprising the inorganic nanoimaging agent(s), and instructions for use of the inorganic nanoimaging agent(s) and/or the composition(s) for carrying out a method of the present disclosure (e.g., a method according to any one of Statements 1-33).
Statement 37. A kit according to Statement 36, where the inorganic nanoimaging agent(s) are chosen from aluminosilicate core-organic ligand shell nanoparticles, each of the aluminosilicate core-organic ligand shell nanoparticles comprising an aluminosilicate core, one or more reference dye group(s) covalently bound to and encapsulated in the aluminosilicate network of the aluminosilicate core-organic ligand shell nanoparticle, one or more sensing dye group(s) capable of analyte sensing covalently bound to the aluminosilicate core network, where the one or more reference dye group(s) and the one or more sensing dye group(s) do not interfere with each other and/or one or more sensing dye group(s) capable of analyte sensing is/are disposed on at least a portion of or all of a surface or at least a portion of or all of the surfaces of the aluminosilicate core, and a plurality of PEG groups disposed on at least a portion of a surface or all of the surfaces of the aluminosilicate core; aluminosilicate core-aluminosilicate shell-organic shell nanoparticles, each of the aluminosilicate core-aluminosilicate shell-organic shell nanoparticles comprising an aluminosilicate core, one or more reference dye group(s) covalently bound to and encapsulated in the aluminosilicate network of the aluminosilicate core, an aluminosilicate shell disposed on at least a portion of or all of a surface or at least a portion of or all of the surfaces of the aluminosilicate core, one or more sensing dye group(s) capable of analyte sensing covalently bound to and encapsulated in the aluminosilicate network of the aluminosilicate shell, optionally, one or more sensing dye group(s) capable of analyte sensing disposed on at least a portion of or all of a surface or a portion of or all of the surfaces of the aluminosilicate shell, and a plurality of PEG groups disposed on at least a portion of a surface or all of the surfaces of the aluminosilicate shell; and any combination thereof.
Statement 38. A kit according to Statement 36 or 37, where the inorganic nanoimaging agent(s) individually have at least one dimension (which may be a longest linear dimension, such as, for example, a diameter) of about 1 to about 30 nm, including all 0.1 nm values and ranges therebetween (e.g., about 2 nm to about 10 nm).
Statement 39. A method of imaging of one or more bone(s) or any portion thereof, one or more musculoskeletal tissue(s) or any portion thereof, or any combination thereof in a sample or in an individual or a portion thereof, the method comprising contacting the sample or the individual with a plurality of one or more inorganic nanoimaging agent(s), where each inorganic nanoimaging agent(s) of the plurality of inorganic nanoimaging agent(s) independently comprises one or more dye group(s), one or more sulfur atom group(s), one or more radioisotope group(s), one or more heavy atom group(s), one or more iodide group(s), or any combination thereof, where the dye group(s), the sulfur atom group(s), the radioisotope group(s), the heavy atom group(s), the iodide(s), or any combination thereof, if present, are independently disposed on a surface of an inorganic nanoimaging agent, covalently or non-covalently bonded to a matrix of the inorganic nanoimaging agent; directing excitation electromagnetic radiation into the sample or the individual, thereby exciting at least one of the one or more dye group(s), if present, one or more sulfur atom group(s), if present, one or more radioisotope group(s), if present, one or more heavy atom group(s), if present, one or more iodide group(s), if present, or the combination thereof resulting in excitation and emission of electromagnetic radiation from the one or more dye group(s), if present, one or more sulfur atom group(s), if present, one or more radioisotope group(s), if present, one or more heavy atom group(s), if present, one or more iodide group(s), if present, or the combination thereof; detecting at least a portion of the emitted electromagnetic radiation, the detected electromagnetic radiation having been emitted by the one or more dye group(s), if present, one or more sulfur atom group(s), if present, one or more radioisotope group(s), if present, one or more heavy atom group(s), if present, one or more iodide group(s), if present, or the combination thereof, and processing signals corresponding to the detected electromagnetic radiation to provide one or more image(s) of the bone(s) or the portion thereof, the musculoskeletal tissue(s) or the portion thereof, or the combination thereof in the sample, the individual, or the portion thereof.
Statement 40. A method according to Statement 39, where the inorganic nanoimaging agent(s) is/are chosen from silica nanoimaging agents, aluminosilicate nanoimaging agents, or the like, or any combination thereof.
Statement 41. A method according to Statement 39 or 40, where the inorganic nanoimaging agent(s) is/are chosen from nanoparticles, nanorings, nanocages, or any combination thereof.
Statement 42. A method according to any of the preceding Statements, where the inorganic nanoimaging agent(s) further comprise(s) a plurality of polyethylene glycol (PEG) groups disposed on at least a portion of a surface or all of the surfaces of the inorganic nanoimaging agent nanoparticle.
Statement 43. A method according to any one of Statements 39-42, where the inorganic nanoimaging agent(s) comprise a longest linear dimension of about 2 nm to about 1 micron.
Statement 44. A method according to any of the preceding Statements, where at least a portion of the dye groups(s) is/are organic dye group(s) or a combination of organic dye group(s).
Statement 45. A method according to any of the preceding Statements, where at least a portion the dye group(s) is/are fluorescent dye groups(s), fluorescent protein groups(s), or any combination thereof.
Statement 46. A method according to Statement 44, where the organic dye group(s) is/are chosen from cyanine dyes, rhodamine dyes, coumarin dyes, boron-dipyrromethene (BODIPY) dyes, xanthene dyes, eosin dyes, carbopyronine dyes, methylene blue, fluorescein, Acridine Orange, structural derivatives thereof, any groups derived therefrom, and any combination thereof.
Statement 47. A method according to any one of Statements 39-46, where the heavy atom group(s) is/are chosen from iodine atom, bromine atom, metal ions, structural derivatives thereof, any group derived therefrom, and any combination thereof.
Statement 48. A method according to Statement 47, where the metal ions are Au ions, Ag ions, Pb ions, Ti ions, Bi ions, Pt ions, In ions, Sn ions, Sb ions or Pd ions.
Statement 49. A method according to any one of Statements 39-48, where the contacting is administering an effective amount of one or more composition(s) comprising the inorganic nanoimaging agent(s) to the individual.
Statement 50. A method according to Statement 49, where the administering is subcutaneous administration, intravenous administration, intraarticular administration, or intramarrow administration.
Statement 51. A method according to any one of Statements 39-50, where the electromagnetic radiation is directed into the individual.
Statement 52. A method according to Statement 51, where the electromagnetic radiation is directed into a region, where the region is within the individual.
Statement 53. A method according to any one of Statements 39-52, where the electromagnetic radiation comprises one or more wavelengths at about 400 to about 1700 nm.
Statement 54. A method according to any one of Statements 39-52, where the electromagnetic radiation is provided by one or more laser(s).
Statement 55. A method according to Statement 54, where the electromagnetic radiation is provided by a tunable wavelength laser.
Statement 56. A method according to any one of Statements 39-55, where the directing, detecting, and processing is fluorescence imaging, bioluminescence imaging, or any combination thereof.
Statement 57. A method according to any one of Statements 39-56, where at least a portion of the inorganic nanoimaging agent(s) is/are aluminosilicate nanoimaging agent(s) and the imaging is super-resolution imaging.
Statement 58. A method according to Statement 57, where the image(s) are fluorescence image(s) or a sequence of fluorescence image(s) which can be processed to obtain a super-resolution optical image of the sample, the individual, or the portion thereof.
Statement 59. A method according to any one of Statements 39-58, where the method is an optical super-resolution microcopy method chosen from ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM).
Statement 60. A method according to any one of Statements 39-59, the method further comprising targeting, diagnosing, treating, preventing, or any combination thereof, a current or potential disease, disease state, condition, disorder, side effect, or any combination thereof, in an individual.
Statement 61. A method according to Statement 60, where the sample is a biopsy sample or a resected tissue sample.
Statement 62. A method according to Statement 60 or 61, where the current or potential disease, disease state, condition, disorder, side effect, or any combination thereof, is chosen from infections, cancers, inflammatory conditions, and any combination thereof.
Statement 63. A method according to any of Statements 60-63, where at least a portion of the inorganic nanoimaging agent(s) independently comprises one or more drug group(s), one or more radioisotope group(s), or any combination thereof, and the contacting results in treatment of the individual.
Statement 64. A method according to any one of Statements 39-63, where at least a portion of the inorganic nanoimaging agent(s) independently comprises one or more targeting group(s).
Statement 65. A method according to Statement 64, where the targeting group(s) are chosen from integrins, transmembrane proteins, intracellular signaling proteins, and any combination thereof.
Statement 66. A method according to Statement 60, where the current or potential disease, disease state, condition, disorder, side effect, or any combination thereof is cancer, and the method further comprises one or more chemotherapy treatment(s), one or more radiation treatment(s), one or more photodynamic therapy treatment(s), one or more surgical intervention(s) or any combination thereof.
Statement 67. A method according to determining a presence, an absence, or a concentration of an analyte proximate to or within one or more bone(s) or any portion thereof, one or more musculoskeletal tissue(s) or any portion thereof, or any combination thereof in a sample, an individual, or a portion thereof using one or more inorganic nanoimaging agent(s) comprising contacting the sample or individual with one or more inorganic nanoimaging agent(s) each inorganic nanoimaging agent comprising: one or more reference dye groups(s), where each reference dye group is covalently bound to and encapsulated in a network of the inorganic nanoimaging agent, and/or one or more sensing dye groups(s), where the sensing groups are capable of interacting with the sample, the individual, or the portion thereof, a plurality of polyethylene glycol (PEG) groups disposed on at least a portion of a surface of the inorganic nanoimaging agent; optionally, incubating an inorganic nanoimaging agent(s) with the sample or in the individual; determining a presence, an absence, or a concentration of the analyte in an individual region of a first object plane using ratiometric sensing; localizing with resolution below Abbe's diffraction limit at least a portion of the individual inorganic nanoimaging agent(s); determining the presence, the absence, or a concentration of the analyte at a position of one of the one or more of the inorganic nanoimaging agent(s) using the presence, the absence, or the concentration of the analyte obtained using the ratiometric sensing and the localization of the inorganic nanoimaging agent(s); and optionally, averaging a fluorescence intensity ratio of a desired number of individual inorganic nanoimaging agent(s) in proximity to an individual inorganic nanoimaging agent to assign an average fluorescence intensity ratio to one or more of the individual inorganic nanoimaging agent(s), where an average fluorescence intensity ratio assigned to the individual inorganic nanoimaging agent(s) corresponds to the presence, the absence, or the concentration of the analyte in the sample, the individual, or any portion thereof.
Statement 68. A method according to Statement 67, where the localizing with resolution below Abbe's diffraction limit at least a portion of the individual inorganic nanoimaging agent(s) is in a second object plane, where the second object plane comprises at least a portion of the first object plane.
Statement 69. A method according to Statement 67 or 68, where the determining the presence, the absence, or the concentration of the analyte in the individual region of the first object plane using ratiometric sensing and the localizing with resolution below Abbe's diffraction limit at least a portion of or all of the individual inorganic nanoimaging agent(s) are each carried out using optical super-resolution microscopy (OSRM) imaging.
Statement 70. A method according to any one of Statements 67-69, where the presence, the absence, or the concentration of the analyte in the sample, the individual, or the portion thereof is determined at a position of one or more of the inorganic nanoimaging agent(s).
Statement 71. A method according to any one of Statements 67-70, where the analyte is chosen from hydrogen ions, oxidants, antioxidants, oxygen, reactive oxygen species (ROS), nitric oxide, chloride ions, metals, metal ions, and any combination thereof.
Statement 72. A method according to any one of Statements 67-71, where the analyte is hydrogen ions and a local pH is determined at a position of at least a portion of the inorganic nanoimaging agent(s) in the sample, the individual, or the portion thereof.
Statement 73. A method according to any one of Statements 67-72, where the individual sensing dye group(s) is/are capable of sensing pH, sensing redox status, sensing the presence or absence of oxygen, sensing the presence or absence of reactive oxygen species (ROS), sensing the presence or absence of chloride ions, sensing the presence or absence of nitric oxide, and/or sensing the presence or absence of one or more metal(s) and/or metal ion(s).
Statement 74. A method according to any one of Statements 67-73, where at least a portion of the inorganic nanoimaging agent(s) further comprise one or more targeting group(s), one or more therapeutic group(s), one or more diagnostic group(s), or any combination thereof.
Statement 75. A method according to any one of Statements 67-74, where the method comprises an OSRM method chosen from ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM).
Statement 76. A method according to any one of Statements 67-75, where the inorganic nanoimaging agent(s) individually have at least one linear dimension of about 1 to about 30 nm.
Statement 77. A method according to any one of Statements 67-76, where the individual is a human or a non-human animal.
Statement 78. A composition for imaging one or more bone(s) or any portion thereof, one or more musculoskeletal tissue(s) or any portion thereof, or any combination thereof in a sample or in an individual, comprising a plurality of one or more inorganic nanoimaging agent(s), where each inorganic nanoimaging agent(s) of the plurality of inorganic nanoimaging agent(s) independently comprises one or more dye group(s), one or more sulfur atom group(s), one or more radioisotope group(s), one or more heavy atom group(s), one or more iodide group(s), one or more targeting group(s), or any combination thereof, where the dye group(s), the sulfur atom group(s), the radioisotope group(s), the heavy atom group(s), the iodide group(s), the targeting group(s), or the combination(s) thereof, if present, are independently disposed on a surface of an inorganic nanoimaging agent, covalently or non-covalently bonded to a matrix of the inorganic nanoimaging agent.
Statement 79. A composition according to Statement 78, where the dye group(s) is/are chosen from cyanine dyes, rhodamine dyes, coumarin dyes, boron-dipyrromethene (BODIPY) dyes, xanthene dyes, eosin dyes, carbopyronine dyes, methylene blue, fluorescein, Acridine Orange, structural derivatives thereof, any groups derived therefrom, and any combination thereof.
Statement 80. A composition according to Statement 78 or 79, further comprising one or more pharmaceutically acceptable excipient(s).
Statement 81. A composition according to any one of Statements 78-80, further comprising a concentration of the inorganic nanoimaging agent(s) of about 10 nanomolar to about 100 micromolar.
Statement 82. A composition according to any one of Statements 78-81, where at least a portion of the inorganic nanoimaging agent(s) independently comprises one or more drug group(s), one or more radioisotope group(s), or any combination thereof.
Statement 83. A composition according to any one of Statements 78-82, where the composition is suitable for subcutaneous administration, intravenous administration, intraarticular administration, or intramarrow administration.
Statement 84. An inorganic nanoimaging agent comprising one or more targeting group(s) independently bonded to a matrix of the inorganic nanoimaging agent and/or one or more dye group(s) independently bonded to the matrix of the inorganic nanoimaging agent.
Statement 85. An inorganic nanoimaging agent according to Statement 84, where the matrix is a silicate or aluminosilicate matrix.
Statement 86. An inorganic nanoimaging agent according to Statement 84 or 85, further comprising a plurality of polyethylene glycol (PEG) groups disposed on at least a portion of a surface or all of the surfaces of the inorganic nanoimaging agent(s).
Statement 87. An inorganic nanoimaging agent according to any one of Statements 84-86, where the inorganic nanoimaging agent comprises a diameter of about 1 nm to about 8 nm.
Statement 88. An inorganic nanoimaging agent according to any one of Statements 84-87, where the inorganic nanoimaging agent comprises a diameter of about 7 nm or less.
Statement 89. An inorganic nanoimaging agent of any one of Statements 84-88, where the one or more dye group(s) are independently chosen from RhG, TMR, Cy3, Cy5, Cy5.5, DY782, and CW800.
Statement 90. An inorganic nanoimaging agent according to any one of Statements 84-89, where the one or more targeting group(s) are independently chosen from integrin binding peptide(s), TAT viral peptide(s), and phalloidin targeting group(s).
Statement 91. A kit comprising one or more inorganic nanoimaging agent(s) and/or one or more composition(s) comprising the inorganic nanoimaging agent(s), and instructions for use of the inorganic nanoimaging agent(s) and/or the composition(s).
Statement 92. A kit according to Statement 91, where the inorganic nanoimaging agent(s) are chosen from aluminosilicate core-organic ligand shell nanoparticles, each of the aluminosilicate core-organic ligand shell nanoparticles comprising an aluminosilicate core, one or more reference dye group(s) covalently bound to and encapsulated in the aluminosilicate network of the aluminosilicate core-organic ligand shell nanoparticle, one or more sensing dye group(s) capable of analyte sensing covalently bound to the aluminosilicate core network, where the one or more reference dye group(s) and the one or more sensing dye group(s) do not interfere with each other and/or one or more sensing dye group(s) capable of analyte sensing is/are disposed on at least a portion of a surface of the aluminosilicate core, and a plurality of PEG groups disposed on at least a portion of a surface of the aluminosilicate core; aluminosilicate core-aluminosilicate shell-organic shell nanoparticles, each of the aluminosilicate core-aluminosilicate shell-organic shell nanoparticles comprising an aluminosilicate core, one or more reference dye group(s) covalently bound to and encapsulated in the aluminosilicate network of the aluminosilicate core, an aluminosilicate shell disposed on at least a portion of a surface of the aluminosilicate core, one or more sensing dye group(s) capable of analyte sensing covalently bound to and encapsulated in the aluminosilicate network of the aluminosilicate shell, optionally, one or more sensing dye group(s) capable of analyte sensing disposed on at least a portion of a surface of the aluminosilicate shell, and a plurality of PEG groups disposed on at least a portion of a surface of the aluminosilicate shell; and any combination thereof.
Statement 93. A kit according to Statement 91 or 92, where the inorganic nanoimaging agent(s) individually have at least one linear dimension of about 1 nm to about 30 nm.

The steps of the methods described in the various examples disclosed herein are sufficient to carry out a method of the present disclosure. Thus, in various examples, a method consists essentially of a combination of the steps of the methods disclosed herein. In various other examples, a method consists of such steps.

The following examples are presented to illustrate the present disclosure. They are not intended to be limiting in any manner.

### Example 1

This Example provides, inter alia, inorganic nanoimaging agent compositions and methods of using same.

Intravital imaging methods offer an opportunity to shed light on a critical feature or features of osteocyte mechanobiology. However, studying small targets like integrins in osteocytes in vivo requires the development of novel probes which are small enough to resolve meaningful integrin populations and bright enough to be seen with presently available imaging approaches.

It was expected to establish ultra-small (~5-6 nm diameter) and bright fluorescent silica nanoparticles in intravital imaging for interrogating molecular/protein level dynamics in osteocytes in vivo to evaluate mechanisms of osteocyte mechanobiology which were previously out of reach for in vivo studies. It was expected that αᵥα₃ integrin targeting C'Dots preferentially will target osteocytes in vivo exhibit different dynamics patterns and be cleared slower than non-specific PEG C'Dots.

Use of MPM for Intravital Analysis of Cell Signaling. Osteocyte Ca²⁻ signaling has been monitored in living mouse bone via MPM using the fluorescent GECI GCaMP3. Ca²⁺ signaling in individual osteocytes was measured before, during, and after mechanical loading to quantify the response to a variety of inputs. To overcome some of the signal-to-noise challenges associated with the in vivo bone microenvironment and improve observation of small targets (e.g., integrins), it was expected to combine MPM with an advanced ultrasmall, bright, and photostable fluorescent nanoparticle platform for in vivo imaging. This advancement represents a giant leap forward in intravital imaging of bone cells and provides an avenue to answer fundamental questions about cellular/molecular level components of osteocyte mechanobiology in vivo.

Cornell Prime Dots. Cornell Prime Dots (C'Dots), developed by the Wiesner Lab in the Department of Materials Science and Engineering at Cornell, are ultrasmall fluorescent silica core-poly(ethylene glycol) (PEG) shell (core-shell) nanoparticles that can be surface-functionalized with multiple targeting peptides. The ~5-6 nm size of C'Dots is kept below the cut-off for renal clearance providing favorable biodistribution (BD) and pharmacokinetic (PK) profiles. The one-pot type synthesis of C'Dots in aqueous solutions and associated mechanisms have been described extensively. Additional benefits of using C'Dots include covalent dye encapsulation, preventing dye leaching and resulting in enhanced per dye brightness and photostability as compared to parent free dye in aqueous solution, narrow particle size distributions and high surface chemical homogeneity, and high targeting affinities via multivalent targeting with specific ligands or antibody fragments.

The combination non-linear fluorescent multiphoton imaging with the latest generation of C'Dot optical labels will be used to study integrin dynamics in osteocytes in vivo. To date ultrasmall C'Dots have not been applied to live-cell imaging in bone. Integrins' size is on the nanometer scale and they exist as spot connections along the osteocyte process; in other words, they are extremely small targets. C'Dots offer desirable options for studying integrins in vivo.

Intravital imaging of embedded osteocytes provides the ability to observe cellular/molecular level responses of osteocytes in situ both statically and with tissue level loading in real time. Initial use of this method focused on bulk cytosolic Ca²⁺ signaling, and use of available fluorescent probes for intravital investigation of osteocytes is expected. This key jump forward directly creates the ability to ask fundamental questions about osteocyte mechanobiology with higher cellular component specificity and, thus, better understand changes in molecular signaling behavior in health and disease. An intravital imaging protocol will be combined with next generation sensors to interrogate osteocyte integrin dynamics in vivo and local Ca²⁻ signaling phenomena.

Current state of the art fluorescent indicators are genetically encoded fluorescent probes, offering a wide range of colors and targets. Their principle advantage is being constitutively produced by target cell populations. C'Dots are brighter and more photostable than genetically encoded indicators and equally versatile in their functionality. The major difference is that they can be delivered locally and cleared via the kidneys without adverse effects, providing for acute delivery of intravital fluorescent probes. Local administration routes of fluorescent agents that are generally cleared within 96 hours will be developed. This is expected to provide the ability to do intravital experiments without transgenic mice and the potential for delivery of multiple sensors at one time or over time. Furthermore, by no longer relying on transgenic approaches to deliver reliable fluorescent probes it becomes possible to conduct intravital imaging experiments in larger animal models.

Intravital imaging is a powerful tool for studying cell and molecular level events in mouse models. Observing cellular dynamics in vivo offers insight into normal physiology and disease progression with surrounding tissue architecture, blood supply, and neighboring tissue interactions intact. Fluorescent markers are a key tool for intravital imaging. Presently a suite of genetically encoded indicators serves as functional and structural markers, however there are limitations to their utility. Transgenic mouse lines take time are very expensive. Moreover, the optical properties of genetically encoded indicators are limited to those presently achieved by EGFP molecules.

C'Dots contain a fluorescent dye (e.g., Cy5) encapsulating silica core and short PEG ligand shells. The particles are several times brighter than parent free Cy5 in water, small enough to be cleared via kidney excretion (~5-6 nm), and highly flexible in application. As examples, they can be surface functionalized to bind to specific proteins and indicate pH or fluctuations in Ca²⁺ concentration. We expect to establish C'Dots for use in an intravital mouse imaging model consisting of in vivo loading and MPM bone cell imaging to study αᵥα₃ integrin dynamics.

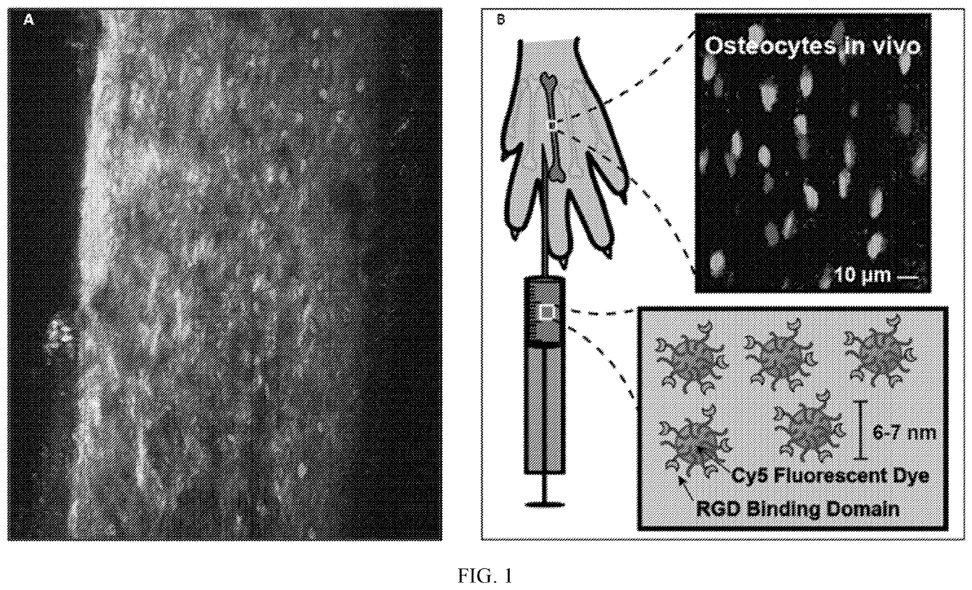

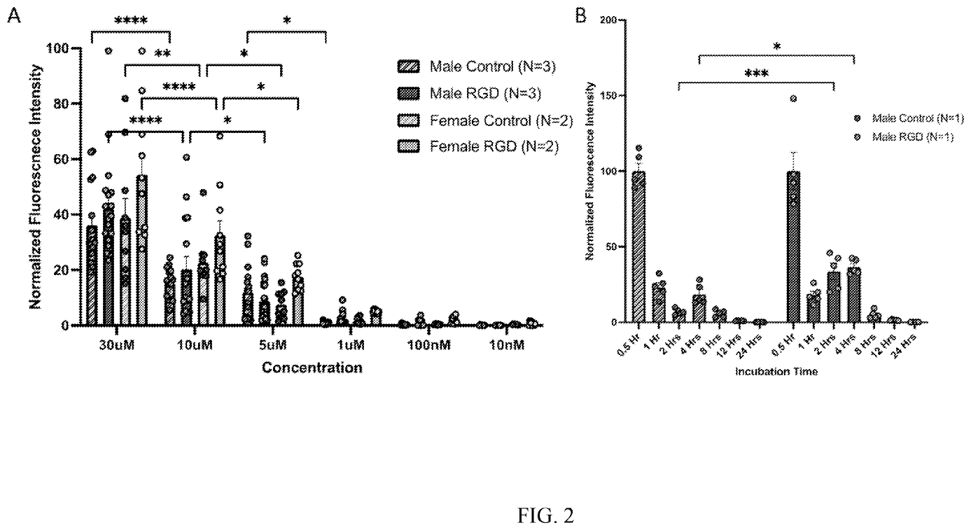

To establish parameters for local injection of C'Dots for imaging in the mouse third metatarsal, different concentrations in 15 μL bolus injections were injected subcutaneously. C'Dots were found to robustly label the osteocytes (FIG. 1). It found that C'Dot signal saturates at 30 μM, and decreases to background at 1 μM (FIG. 2A). These data suggest that a useful working solution would be between 5-10 μM for future experiments. A preliminary time course experiment was then performed using 10 μM concentration in 15 μL injections. Times between 0.5-24 h were interrogated. It was found that for both untargeted C'Dots and αᵥβ₃ integrin targeting arginine-guanine-aspartic acid (RGD) C'Dots there was a decline in fluorescent brightness in osteocytes over time (FIG. 2B). There is some indication that the temporal dynamics of each C'Dot variant may differ, likely due to integrin interactions with RGD-C'Dots. These data firmly support further stratification of the use of various C'Dot variants for intravital imaging.

Establish local administration of C'Dots for studying osteocyte αᵥβ₃ integrins in vivo. Intravital imaging approaches to study musculoskeletal cells require robust and flexible fluorescent agents. C'Dots are ultrasmall fluorescent silica nanoparticles that are extremely bright, photostable, and small enough to be cleared by the kidney. Indeed, they have been used in several in vitro and clinical applications for visualizing cells. However, their utility for intravital imaging in mouse models has not been established. C'Dot concentration and incubation dynamics will be interrogated in an intravital bone model to establish working parameters for future experiments. It was expected to illuminate the impact of functionalizing the surface of C'Dots with integrin targeting RGD sequence.

For all experimental groups, C'Dots will be locally administered to mouse third metatarsal bones via subcutaneous injection of the hind paw immediately above the bone. Groups will receive either C'Dots, RGD-C'Dots, or scrambled RGD-C'Dots along with calcein-AM. Colocalization of C'Dot Cy5 signal and calcein signal will be used to establish whether C'Dots are located intracellularly or in the external microenvironment. A concentration dose response protocol ranging from 10 nM-30 μM with an incubation time of 1 hour will be used to determine the minimum concentration appropriate for local administration. Next, a time course experiment will be conducted using the arrived at concentration where incubation times will be varied between 0.5-24 hours to determine the temporal dynamics of each C'Dot variant (n=10/group/sex/experiment).

Once appropriate dose and incubation times are established, an in vivo loading experiment will be conducted to determine the load induced dynamics of integrin targeting C'Dots. Groups will receive either C'Dots, RGD-C'Dots, or scrambled RGD-C'Dots along with calcein-AM. C'Dots will be delivered via subcutaneous injection directly over the third metatarsal, followed by incubation with normal activity. Third metatarsals in each experimental group will be loaded in a strain dose response experiment (n=10/group/sex). Briefly, the third metatarsal of anesthetized mice will be surgically exposed and positioned into an in vivo loading device. The bones will be loaded at 1 Hz to calibrated strains of 1000 με for a total of 5 bouts per mouse. During loading simultaneous intravital multiphoton imaging of osteocyte fluorescent signal will be performed. Volumetric images will also be collected between each loading bout to provide a temporal assessment of changes in overall cellular fluorescence intensity.

Based on preliminary data, it is expected that a desired concentration to fall between 5-10 μM. Moreover, the targeted RGD-C'Dots is expected to present intracellularly and take longer to clear from the osteocyte microenvironment than non-targeted C'Dots. Loading will increase the amount of RGD associated fluorescent signal and prolong its presence. Differences between sexes are not expected.

Untargeted and targeted RGD-C'Dots will be injected with the expectation that they will be cleared at different rates form the osteocyte microenvironment. The idea is that targeted RGD-C'Dots will be bound to cells and therefore be more temporally stable, and potentially endocytosed at a higher rate. If this does not occur, the versatile nature of C'Dots can be exploited and other candidates found for surface functionalization to slow clearing and increase cellular uptake.

Determine the utility of ratiometric C'Dot Ca²⁺ sensing in vivo. C'Dots equipped with ratiometric Ca²⁺ sensing properties offer a promising alternative to GECIs in intravital imaging. Their material properties increase fluorescence intensity of the Cy5 core several times. This enhanced brightness paired with their diminutive size (<8 nm diameter) make them ideal sensors for intravital imaging. The performance of GCaMP6f and ratiometric Ca²⁺ sensing C'Dots will be compared in intravital imaging of load induced osteocyte Ca²⁺ signaling. The so-called osteocyte mechanosome is a collection of channels clustered near αᵥβ₃ integrin attachment sites. Understanding Ca²⁺ signaling events near integrin attachments offers key improvement in the granularity of information about changes in osteocyte mechanotransduction events in health and disease. To this effect, will also compare integrin targeting RGD Ca²⁺ sensing C'Dots with non-targeted PEG Ca²⁺ sensing C'Dots.

GCaMP6f expression will be achieved in osteocytes by crossing Ai95 mice with DMP-1/Cre mice (Jackson Labs). Ratiometric Ca²⁺ sensing C'Dots will be delivered via subcutaneous injection directly over the third metatarsal, followed by 1-hour incubation with normal activity. Third metatarsals in each experimental group will be loaded in a strain dose response experiment (n=10/group/sex). Briefly, the third metatarsal of anesthetized mice will be surgically exposed and positioned into an in vivo loading device. The bones will be serial loaded at 1 Hz to several calibrated strains ranging from 250-2000 με, spanning the physiological strain range. During loading simultaneous intravital multiphoton imaging of osteocyte fluorescent Ca²⁺ signaling events will be performed. The number of responding cells (denoted by a >20% change in fluorescence intensity over baseline) will be counted and the average response intensity among responding cells. The number of responding cells, dynamic range, baseline brightness, and peak brightness will be compared between groups.

Expected Outcomes: Ca²⁺ sensing C'Dots will have higher baseline fluorescence and dynamic range compared to GCaMP6f. Moreover, the number of responding cells is expected to be similar between experimental groups, indicating the utility of C'Dots to be used in place of GCaMP6f for Ca²⁺ sensing experiments in vivo. Enhanced Ca²⁺ sensing C'Dot signal is expected compared to controls, and intensity fluctuations near clusters of integrin attachments. Ca²⁺ signaling events are expected to be made out both at the process and cell body.

C'Dots, like other injected fluorescent markers, are eventually cleared from cellular environments. As such, ratiometric C'Dot fluorescent signal may decrease over time. Data indicate relatively consistent presence of non-functionalized C'Dots out to 4 hours post-injection, so we do not expect their signal to decrease within the scope of this proposed experiment. However, if a decline is noticed, the incubation period can be shortened to 1 hour.

Establishing C'Dot use for intravital interrogation of osteocyte integrin dynamics provides the opportunity to ask questions about cellular dynamics and molecular metabolism in vivo that were previously unavailable. With this approach, insight into a key feature of bone metabolism is expected to be provided and the means to better understand how mechanosensation changes in osteocytes in vivo with disease created.

Experimental Methods and Procedures. Mice—For C'Dot experiments, C57BL/6 mice will be used at 16-18 wks of age (n=10/group/sex, Jackson Labs). Mice exhibiting osteocyte-targeted expression of the GECI GCaMP6 are achieved by crossing Ai39 mice (Jackson Labs), which contain GCaMP6 DNA behind a Lox-STOP-Lox codon, with DMP1-Cre mice (Jackson Labs). Initial studies with this model showed expression of an earlier version of this construct, GCaMP3, in essentially all cortical osteocytes. Similar expression is seen in GCaMP6 mice in our hands.

Synthesis of PEGylated fluorescent core-shell silica nanoparticles (C'dots). The synthesis of C'Dots uses well-established protocols in water as reaction medium. In a first step, Cy5-maleimide (Cy5(+), Lumiprobe) is conjugated in DMSO (Sigma Aldrich) with a 25-fold excess of mercapto-trimethoxy-silane (MPTMS, Millipore Sigma) to form a Cy5-silane conjugate overnight. This conjugate is then added to 10 ml of slightly basic pH(~8) aqueous solution (adding ammonia solution in ethanol as a base, Millipore Sigma) together with tetramethyl orthosilicate (TMOS, Millipore Sigma) as silica precursors at a molar ratio of ~1:1000 and left stirring overnight at room temperature. The particle synthesis is finalized by adding PEG6-9-silane (2-[methoxy (polyethyleneoxy) 6-9propyl]trimethoxysilane, Gelest) (23 nM) to the solution dropwise. After stirring overnight, the reaction mixture is heated to 80° C. for 24 h without stirring.

Synthesis of RGD/scrambled RGD (control) surface functionalized C'Dots. The synthesis of RGD or scrambled RGD (used as controls) functionalized C'Dots proceeds in the same way as described for the synthesis of regular C'Dots, except for the PEGylation step, again following well-established protocols. For PEGylation, heterobifunctional PEG with maleimide and NHS-ester groups (mal-PEG12-NHS, molar mass ~870, Quanta BioDesign) is first conjugated with (3-aminopropyl)-triethoxysilane (APTES, Millipore Sigma) in DMSO at room temperature under nitrogen overnight to form a mal-PEG-silane. Cyclo (arginine-glycine-aspartic acid-D-tyrosine-cysteine), c(RGDyC), or scrambled RGD, i.e. c(RADyC), peptide (Peptide International) is then added to this DMSO solution and left overnight under nitrogen (molar ratio c(RGDyC):mal-PEG12-NHS:APTES is 1.1:1.0:0.9). In the PEGylation step during nanoparticle synthesis, the produced c(RGDyC)-PEG12-silane (or c(RADyC)-PEG12-silane) is added dropwise to the particle growth solution, followed by the addition of PEG6-9-silane. The molar ratio of c(RGDyC)-PEG12-silane to PEG6-9-silane can be used to vary the amount of peptide ligand per particle.

Synthesis of ratiometric C'dot/aC'dot Ca²⁺ sensors. The synthesis of C'Dot/aC'Dot ratiometric sensors is conducted using well-established protocols described earlier. After the 80° C. heating steps in the C'Dot/aC'Dot syntheses, the solution is purified via GPC as described below. Following an approach established as post-PEGylation surface modification by insertion, APTMS is first added to the reaction mixture and stirred overnight. DBCO-PEG4-NHS ester (Click Chemistry Tools) is then added and stirred overnight. The C'Dot/aC'Dot solution is then purified via GPC as described below. The number of DBCO's per C'Dot/aC'Dot is then determined by fluorescence correlation spectroscopy (FCS) and UV-Vis spectroscopy as described in previous publications. Based on this analysis, Ca sensor dye with azide conjugation group (Calbryte™-520XL azide, AAT Bioquest) is then added to the DBCO-functionalized particles at a 10:1 sensor dye:dot molar ratio. This amount may be adjusted based on the desired number of sensor dye molecules per C'Dot/aC'Dot. Particles are then submitted to final purification steps as described below.

Particle purification and characterization. Particle purification and characterization steps follow well-established published protocols. Purification. After cooling heated solutions back to room temperature, particle solutions are dialyzed with 2 L of Milli-Q 18.2MΩ H₂O with 3 exchanges. Particle solutions are then syringe filtered across a 0.22 μm PVDF filter (Foxx Life Sciences) and subsequently up-concentrated in a 30 kDa spin filter (Corning) at 24,000 g. Particles are size purified using a GPC (Bio-Rad) with 0.9% NaCl (Santa Cruz Biotechnology) running solvent. The fractions that elute within the full width at half maximum (FWHM) of the resulting chromatogram are collected and resuspended in 18.2MΩ H₂O. Characterization. Particle characterization includes fluorescence correlation spectroscopy (FCS) for hydrodynamic size/diameter (HD), particle concentration, and particle brightness determinations, a combination of FCS and UV-Vis to assess number of dyes and specific ligands per particle as well as quantum efficiency enhancement relative to free dye, transmission electron microscopy (TEM) to determine silica core size, zeta-potential measurements to quantify particle surface charge, gel permeation chromatography (GPC) to assess particle purity, and high-performance liquid chromatography to determine surface chemical heterogeneities.

Metatarsal C'Dot Injection—Mice (16-18 wks) will be anesthetized using isoflurane (2-3% at 1 L/min) and positioned in a supine posture. C'Dot solutions will be injected in 15 μL volumes with a Hamilton syringe subcutaneously over the third metatarsal. The syringe is angled at 20° and slowly depressed to allow time for the fluid to fill the subcutaneous space without pressurizing and ejecting. Mice are injected with either c(RGDyC)-Cy5(+)-C'Dots or PEG-Cy5(+)-C'Dots according to experimental group. All injection volumes include 30 μM calcein-AM, which provides green intracellular fluorescent signal.

Intravital Imaging and Analysis—Multiphoton imaging will be used to view fluorescent signals through cortical bone in MT3 osteocytes of anesthetized mice. 0.5-2% isoflurane will be vaporized with medical grade air and administered at 1 L/min. Mice will be kept under anesthesia for no longer than 4 hours at a time, warmed with a heating pad, and monitored with a pulse oximeter. Fluorescent signals will be visualized using a broadly tunable Ti:Sapphire laser with internal total power control (Discovery NX, Coherent) paired with resonant galvo optics (Bergamo II, Thorlabs) and a 20× water immersion lens (XLUMPLFLN, Olympus). Cy5 laden C'Dots will be imaged with 1090 nm excitation and 647 nm long pass filter acquisition. Calcein-AM and GCaMP6 will be imaged using 920 nm excitation and 520±30 nm bandpass emission.

For static image analysis 30 μm Z-stacks (0.3 μm steps) will be acquired starting ~20 μm below the bone surface with a full frame field-of-view of 602×602 μm and 1024×1024 resolution. C'Dot fluorescence intensity measurements from individual osteocytes will be taken by processing the z-stacks in ImageJ (NIH). A macro was created to interpolate the regions of interest across all layers of the z-stack and well as filtering with despeckling and a Gaussian blur. Then 3D segmentation is run, creating objects for all cell volumes over a threshold intensity. The lower bound for threshold intensity is set as 3× SD between background fluorescence of different experimental groups. These objects are filtered by surface area (>1000 pixels) to ensure only real cell volumes were counted, and then mean intensity for each object is quantified.

For acquiring real-time imaging with dynamic mechanical loading, single plane images will be acquired at a frequency of 10 Hz approximately 20 μm from the bone surface with a full frame field-of-view of 602×602 μm and 512×512 resolution. Recordings will be collected for 150 s; 60 s pre-load, 60 s loading, and 30 s post-load. Cells within the volume will be analyzed with individual ROIs and normalized to their average baseline (pre-load) fluorescence intensity. A responsive cell will be one whose fluorescence intensity increases by >20% over baseline. The number cells which respond to load and their average response intensity will be calculated for each recording. Each mouse will receive 8 loading bouts, 2 for each of the 4 strain levels.

Metatarsal Loading with Intravital Imaging—Osteocyte Ca²⁺ signaling in response to mechanical loading in vivo will be examined in mouse third metatarsal (MT3) diaphysis of GCaMP6 mice with an in vivo loading model. MT3 bones are loaded in 3-point bending at 1 Hz to mid-diaphyseal strain levels of 250, 500, 1000, 2000 με. These levels encompass the range of strains that have reported during physiological activities from in vivo strain gage studies, with strains up to 2000 με characteristic of habitual activities. All loading procedure are carried out in mice anesthetized with isoflurane. First, the MT3 dorsal surface is accessed by a single scalpel incision through the skin and extensor aponeurosis, with care taken to isolate and avoid the dorsal foot arteries. The lower stainless-steel 1 mm diameter cylindrical fulcrum pin of the 3-point bending loading device is inserted beneath the MT3 mid-diaphysis, thus functionally isolating the bone. The pin is placed in its anchor bracket and the entire foot positioned in the 37° C. PBS bath of the loading apparatus. The upper stainless steel loading contact points are then positioned on the dorsal bone surface and a nominal tare strain (~100 με) is applied to prevent the bone from moving during loading. The entire apparatus is set onto the stage of a multiphoton microscope. Bones are loaded cyclically under displacement control as described about (D.7); coefficients of variation for each target strain level were previously determined to be ~15%. The duration of each strain-loading bout is 60 s and is performed with imaging. The loading bouts are followed by 15 min of rest. The loading and rest procedure is repeated for the next strain level.

Data Analysis—All image processing of multiphoton microscopy data will be handled with ImageJ (NIH) or MATLAB. Intensity changes through the depth of z-stacks will be measured as average pixel intensity for matrix and cell laden volumes separately. Changes in fluorescence intensity for each type of signal will be compared using a students-t test. Number of responding cells and response intensity of responding cells will be analyzed using a two-way analysis of variance, with a Tukey post-hoc test. Statistical analyses will be performed with R, MATLAB, or PRISM. Statistical analyses will be performed with R, MATLAB, or PRISM.

### Example 2

This Example provides, inter alia, inorganic nanoimaging agent compositions and methods of using same.

Pharmacological perturbation of select endocytosis pathway components was used to interrogate trafficking of fluorescent nanoparticles in osteocytes by visualizing uptake, subcellular localization, and clearance kinetics. Specifically, dynamin GTPase activity (implicated in clathrin endocytosis) and cholesterol-based lipid rafts (implicated in caveolae endocytosis and micropinocytosis) were targeted. Our novel intravital imaging studies successfully demonstrate that nanoparticle uptake and clearance in osteocytes can be modulated by small molecule endocytosis disruptors. Results show that pre-incubation with dynamin-inhibitor Dyngo4a significantly increases C'Dot signal intensity and retention in the males while pre-incubation with cholesterol-inhibitor MβCD results in a significant increase in the female group. These data suggest that osteocyte endocytic pathways, specifically receptor mediated endocytosis of integrins, are differentially regulated between the sexes.

Two broad-acting small molecule endocytosis pathway disruptors were used to target swaths of potential innate osteocyte endocytosis and protein trafficking mechanisms. Dyngo4a is a potent dynamin inhibitor derived from Dynasore that blocks dynamin G domain GTPase activity and perturbs clathrin-mediated and other endocytic pathways. Methyl-β-cyclodextrin is a cholesterol and lipid raft depleting agent that is known to disturb caveolae-mediated endocytosis. Together, these drugs impact most forms of endocytic pathways, and in the following experiments, how a local, subcutaneous injection of these broad-acting drugs impacts osteocyte metabolism and trafficking of C'Dot nanoparticles in vivows assessed.

Methods. Animals: Male and female 16-20 week old C57Bl/6J mice (Jackson Laboratory, Bar Harbor, ME) were used in these experiments, representing skeletally mature young adult mice. All procedures were approved by the Institutional Animal Care and Use Committees at Cornell University.

Synthesis of cRGD and PEGylated Silica Core-Shell Nanoparticles: Ultrasmall fluorescent core-shell silica nanoparticles with encapsulated Cy5 dye were synthesized by a modified Stöber process in water as previously described. Two varieties, untargeted control (PEG-C'Dots and integrin targeted (RGD-C'Dots) C'Dots, were synthesized as previously reported.

C'Dot Injection and Incubation: Mice were anesthetized with 2-3% isoflurane mixed with medical grade air in an induction chamber for 3 minutes prior to injection. During injection, mice were kept under 2% isoflurane with a nose cone. A 10 μL subcutaneous injection of 10 μM C'Dots was then administered over the third metatarsal (MT3). C'Dots were incubated for 1 hour prior to MT3 isolation (n=4-5/group/sex). Mice were allowed normal cage activity during their incubation time, and were re-anesthetized immediately before MT3 isolation.

Metatarsal Isolation Surgery: MT3 bones were isolated as previously described. Briefly, while mice were anesthetized, a shallow vertical incision was made between the second and third metatarsal of the mouse hind paw and overlying tendons were removed. The MT3 was functionally isolated from the rest of the paw with a stainless steel pin beneath the mid-diaphysis of the bone, leaving primary vasculature at the proximal and distal palmar aspects intact. The bone was then stabilized in a 3-point bending configuration and submerged in room temperature DPBS prior to imaging. Mice were continuously anesthetized at 1.5-2% isoflurane during all subsequent imaging.

C'Dot Imaging: C'Dot fluorescent signal inside the MT3 was visualized with multiphoton microscopy (MPM) (Bergamo II, Thorlabs) as previously described (Matthews 2023). Briefly, a 20× immersion objective (XLUMPLFLN, Olympus), 1090 nm wavelength excitation, and a >647 nm longpass filter acquisition were used for Cy5 encapsulated C'Dots. Images were captured at 1024×1024 pixel density and 0.588 μm pixel resolution. For the C'Dot clearance study, a 35 μm z-stack was taken for each mouse, starting 20 μm beneath the bone surface, with 0.3 μm steps between each slice and 7 frame averaging per slice. These z-stacks were taken every 15 minutes for almost 3 hours after the initial 1 hour incubation.

C'Dot Image Analysis: Z-stacks were analyzed in ImageJ (NIH) as previously described (Matthews 2023). Briefly, a macro was created to interpolate the regions of interest across the z-stack as well as filter the images. Then 3D segmentation was run, creating objects for all cell volumes over a threshold intensity. The lower bound for threshold intensity was established as the mean plus 3× standard deviation of background fluorescence. Segmented objects were then filtered by surface area (>1000 pixels) to ensure only full cell volumes were counted, and mean intensity for each object was quantified using the 3D manager tool. Intensities were normalized to background within each z-stack.

Quantification of C'Dot Subcellular Localization: Individual frames from z-stacks taken after a 45 minute C'Dot incubation were analyzed for subcellular localization within osteocytes under different study conditions. Filtering with a gaussian blur was performed prior to thresholding of signal and particle analysis to identify cellular regions of interest (ROIs). These ROIs were overlayed onto the original image. Cells with distinct subcellular localization of C'Dots were counted as a percentage of total cells. Other cells had saturated or non-distinct signal. Cells with C'Dot signal in with a rim phenotype were also quantified by-eye. Two blinded individuals quantified the images and averaged consensus data was used in the final results.

Endocytosis Inhibition Experiment: The small molecule Dyngo4a was used as a potent Dynasore analogue to evaluate the impact of dynamin-mediated endocytosis inhibition. A pre-incubation of Dyngo4a (10 μL, 30 μM) was subcutaneously injected above the MT3 30 minutes prior to C'Dot injection and subsequent imaging. Dyngo4a was diluted from a stock solution in DMSO to the working concentration in 1×PBS. Dyngo4a was kept at 20 C and was defrosted prior to use. Methyl-β-cyclodextrin (MβCD) was used as a depletor of plasma membrane cholesterol to evaluate the impact of cholesterol-mediated, lipid raft-based endocytosis inhibition. A pre-incubation of MβCD (10 μL, 10 mM) was subcutaneously injected 30 minutes before C'Dot injection. MβCD was diluted from a powder in 1×PBS. MβCD was kept at 4 C and warmed to room temperature prior to use. Both drugs were tested across all groups: male and female, PEG- and RGD-C'Dots (n=4-5/group/sex).

Statistical Analyses: Clearance study analysis was done using a student's t-test, 2-way ANOVAs, and regression analyses, including non-linear (One Phase Decay) and linear. Analyses were performed in GraphPad Prism version 9.0 for Mac OS X (GraphPad Software).

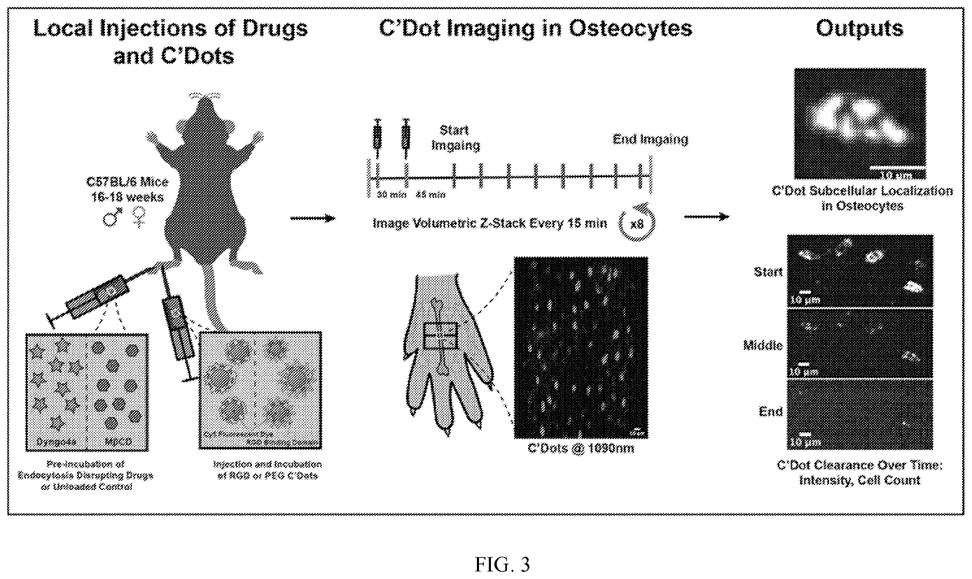

Results. Local Delivery of Endocytosis Small Molecule Disruptors to Osteocytes in vivo. Osteocytes in C57Bl/6J murine metatarsal long bone were exposed to Dyngo4a and MβCD small molecule drugs via a local subcutaneous injection, as previously described for C'Dot injection. These drugs were assessed for their ability to modulate osteocyte metabolism and trafficking. Previously validated fluorescent nanoparticles were used to observe changes to osteocyte activity in vivo. Either untargeted PEG-C'Dots and integrin-targeted RGD-C'Dots were injected into the mouse hind paw after preincubation with Dyngo4a or MβCD to understand differences in drug impact on targeted or untargeted nanoparticle metabolism (FIG. 3). Volumetric multiphoton near-infrared imaging (1090 nm excitation) of C'Dot signal over time was collected and quantified to observe drug induced changes in osteocytes. Outputs from MPM imaging include subcellular localization and clearance kinetics.

### Impact of Dyngo4a on Osteocyte Nanoparticle Clearance

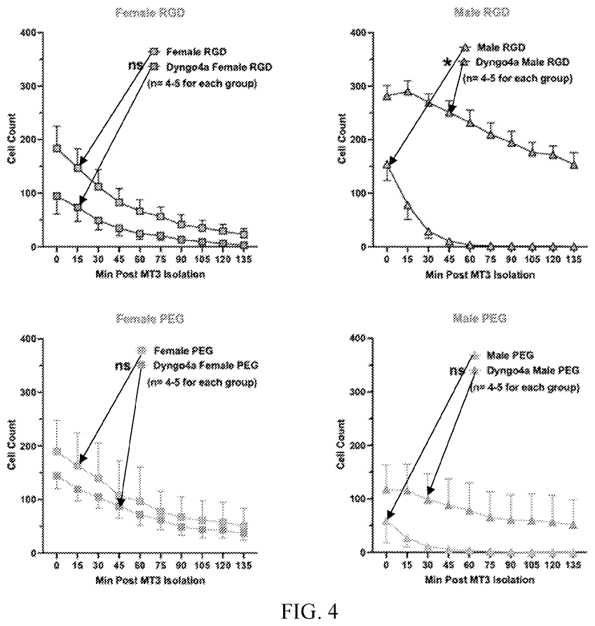

After a 30 minute preincubation of the dynamin-mediated endocytosis disrupting drug (Dyngo4a) and 45 minute incubation of C'Dots, clearance experiment imaging was performed every 15 minutes for 2.5 hours and compared to untreated control C'Dot mice (previously reported) to confirm changes in signal. Our first assessment confirmed that the small molecule drug was able to reach osteocytes in vivo and impact their normal metabolic activity. The number of cells visualized and quantified at the initial imaging timepoint after Dyngo4a preincubation and metatarsal isolation was significantly higher in male mice with RGD-C'Dots compared to untreated control while there was no statistically significant impact in females (FIG. 4, p≤0.05, 2-Way ANOVA). Females even appeared to have diminished signal compared to untreated control C'Dot clearance. These patterns were replicated with PEG-C'Dots but differences were not as large, with neither males or females having significant change from untreated control mouse clearance. PEG groups also had more inter-animal variation, resulting in sizeable error bars as compared to RGD groups.

Retention of RGD-C'Dot signal in male mice was significantly increased compared to untreated controls as well (p≤0.05, 2-Way ANOVA). While untreated males lost signal after only an hour of imaging, Dyngo4a caused signal to be maintained until the end of the imaging experiment (FIG. 4). For females, RGD-C'Dot signal with Dynog4a preincubation was near zero by the end of the experiment. These results clearly confirm sexual dimorphism in osteocyte nanoparticle metabolism. Dynamin appears to be necessary for normal RGD-C'Dot clearance only in male mice.

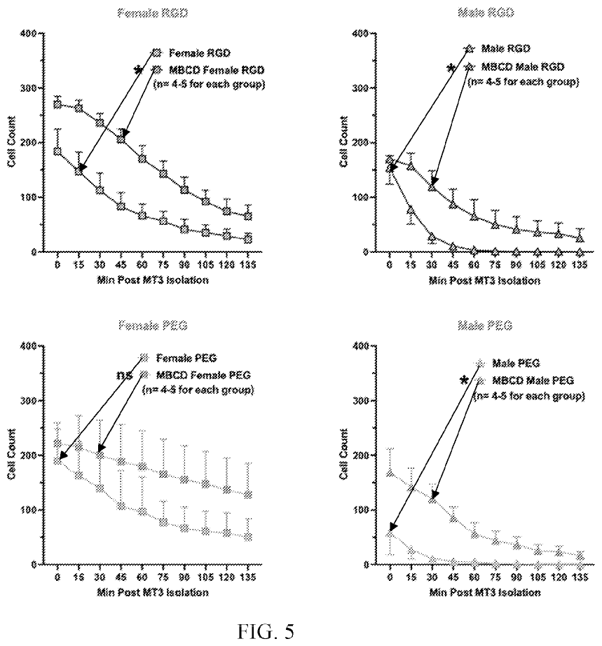

Impact of MβCD on Osteocyte Nanoparticle Clearance. After the impact of dynamin-mediated endocytosis disruption was assessed, the response of osteocytes to a different type of endocytosis disruptor, MβCD was tested. This small molecule cholesterol depletor was preincubated for 30 minutes as well prior to C'Dot application and subsequent bone exposure and imaging. It was found that pre-incubation with MβCD resulted in a significant increase in RGD-C'Dot signal in both the female and male groups (FIG. 5, p≤0.05, 2-Way ANOVA). The male RGD signal was much lower compared to the increase seen with Dyngo4a. PEG C'Dot groups again has similar patterns in clearance, however only males retained a significant increase in signal compared to untreated control. The female PEG group again exhibited large inter-animal variation. These results suggest that cholesterol is necessary for normal RGD-C'Dot metabolism in both male and female mice as well as untargeted C'Dot clearance in males.

### Regression Analysis of C'Dot Clearance Under Untreated and Drug Conditions

Regression analyses were performed to understand how endocytosis disrupting drugs impacted the slope and initial C'Dot signal uptake in osteocytes. Simple linear regression had fits of 0.64 and 0.52 for male and female RGD groups respectively (Table 1). PEG group fits were lower at 0.22 and 0.35 respectively. Using linear regression lines, slopes were significantly different between the male RGD-C'Dot group and both Dyngo4a and MβCD clearance studies. The same was true for female RGD-C'Dot groups. These results suggest that endocytosis disrupting drugs impact the rate of C'Dot clearance and nanoparticle metabolism in osteocytes. For the RGD-C'Dot groups, differences in y-intercepts were unable to be calculated because the lines were already significantly different.

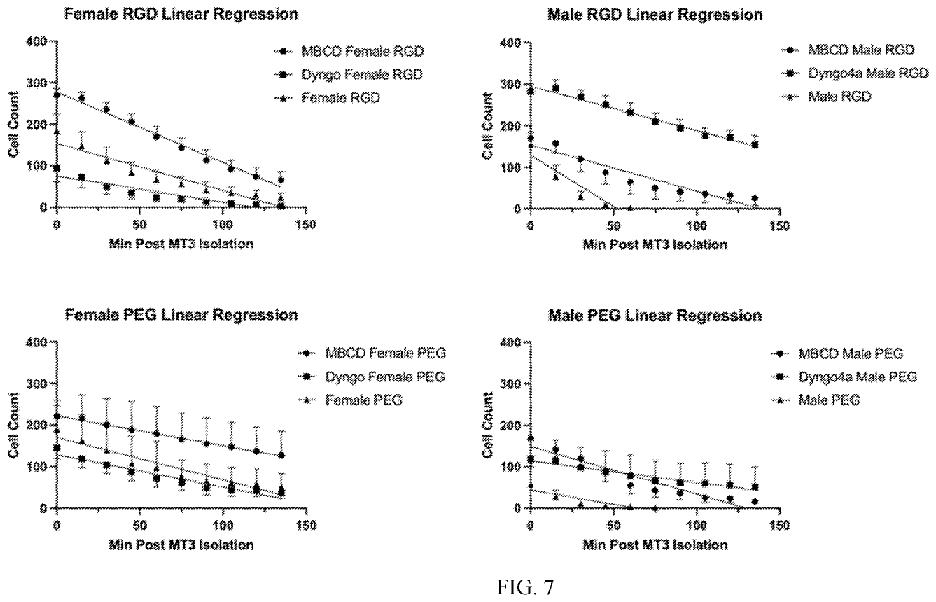

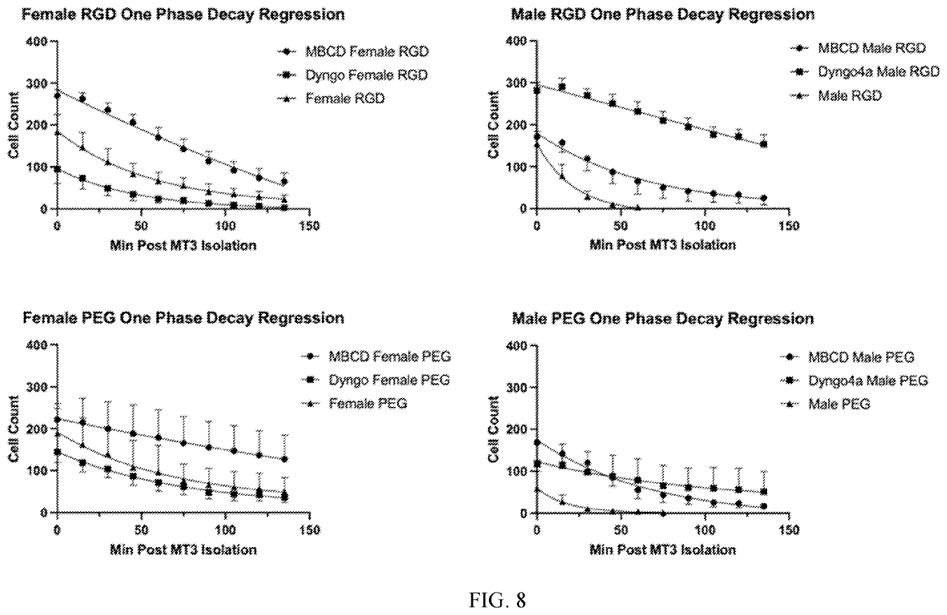

Table 1: Regression Results comparing differences between C'Dot Clearance lines under different endocytosis drug pre-incubation conditions. Upper (Red) section uses simple linear regression. Lower (Blue) section uses One Phase Decay regression. Bolded sections show significant differences (P≤0.05). Graphs with regression lines shown in supplemental data (see e.g., FIG. 7 and FIG. 8).

| C'Dot | Linear | Linear | Linear Y- | Linear | Linear Y- |
|---|---|---|---|---|---|
| Treatment | Regression | Slope vs | Intercepts | Slope vs | Intercepts |
| Group | (Fit − R\hat{ }2) | Dyngo4a | vs Dyngo4a | MBCD | vs MBCD |
| Male RGD | 0.6438 | P = 0.0036 | — | P = 0.0134 | — |
| Male PEG | 0.2191 | P = 0.8239 | P = 0.0009 | P = 0.2527 | P < 0.0001 |
| Female RGD | 0.5249 | P = 0.0148 | — | P = 0.0134 | — |
| Female PEG | 0.3457 | P = 0.3853 | P = 0.0431 | P = 0.5691 | P = 0.0025 |

| C'Dot | One Phase | One | One |
|---|---|---|---|
| Treatment | Decay | Curve | Curve |
| Group | (Fit − R\hat{ }2) | w/Dyngo4a? | w/MBCD? |
| Male RGD | 0.7395 | No <0.0001 | No <0.0001 |
| Male PEG | 0.2849 | No 0.0163 | No <0.0001 |
| Female RGD | 0.5838 | No <0.0001 | No <0.0001 |
| Female PEG | 0.374 | Yes 0.1936 | No 0.0234 |

The slopes of male and female PEG C'Dot groups were not significantly different compared with Dyngo4a or MβCD pre-incubation, suggesting no changes to the rate of nanoparticle clearance. However, the y-intercepts for both comparisons were significantly different, suggesting modulation in the amount of initial cellular uptake of C'Dots, even if the method of clearance is the same. These results indicate that there are differences in uptake and clearance mechanisms between targeted and untargeted nanoparticles that our platform is able to quantify.

One-Phase Decay regression analysis (OPD) was performed to compare a curved to linear regression for fit and significance. OPD fits were improved for all groups compared to the linear regression, specifically with 0.74 and 0.58 for male and female RGD groups respectively (Table 1). This regression tested if a single curve could be used to describe the clearance profile of controls compared to drug groups. Both male and female RGD groups had statistically distinct curves with both Dyngo4a and MβCD (p≤0.0001). PEG groups were also statistically distinct except for the female PEG group compared to Dyngo4a pre-incubation.

Altogether, regression analysis strongly confirms that osteocytes treated with endocytosis disruptors are not the same as untreated controls.

### Quantifying Subcellular Localization of C'Dots

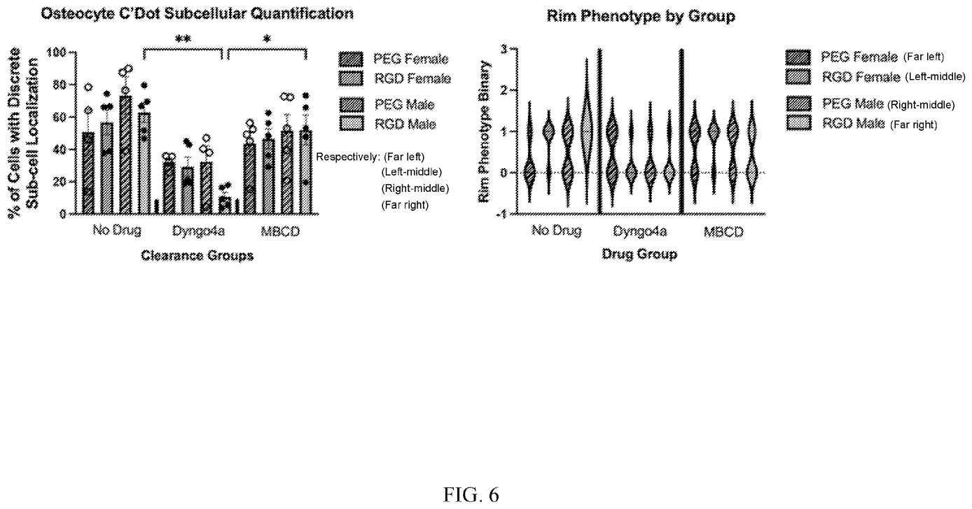

Discrete subcellular localization of RGD C'Dot signal is seen in ~75% of osteocyte cell bodies in untreated controls for both sexes (FIG. 6). Pre-incubation with dynamin inhibitor Dyngo4a reduced the number of cells with discrete localization to 25% in males (ANOVA, p≤0.0001), and had a non-significant decrease in females. Pre-incubation with cholesterol depletor MβCD did not significantly change the percentage of cells with subcellular localization in any group. Rim phenotypes were not modulated in any of the groups under either of the drug pre-incubations.

Discussion Results presented in this paper confirm the ability of small molecule endocytosis disruptors to impact osteocyte activity in murine long bone with a local subcutaneous injection. This establishes C'Dots as a platform and methodology to assess osteocyte mechanosensitivity under different acute endocytic conditions.

Dynamin inhibition experiments indicated that dynamin GTPase activity is critical for rapid clearance and subcellular localization of RGD C'Dots in male mice, but appears to be less impactful for females (FIG. 4). These data suggest that osteocyte endocytic pathways, specifically dynamin-mediated endocytosis of integrins, are differentially regulated between the sexes. Increased variability and reduced impact of Dyngo4a seen in the PEG-C'Dot clearance data supports that integrin specific endocytosis is distinct compared to non-targeted C'Dots. Research into receptor-mediated endocytosis supports this hypothesis. Both endocytic pathways and regulation of those pathways may be mechanisms that cause the observed divergence between our untargeted and receptor (integrin) targeted groups. Other nanoparticles have been shown to enter cells via a dynamin- and clathrin-mediated endocytic pathway²⁸, so it is intriguing that only the integrin-targeted C'Dot clearance profile in male mice was significantly impacted by dynamin inhibition.

The results on the effect of MβCD indicate that cholesterol and lipid raft functionality is critical for initial uptake of both untargeted and integrin-targeted C'Dots and for clearance of integrin-targeted C'Dots within osteocytes in vivo (FIG. 5). This aligns with other research on the nanoparticle uptake and the importance of caveolae-mediated endocytosis for maintaining bone tissue. Caveolae-medicated endocytosis is known to have a negative impact on bone mass; Cav-1 deletion in a mouse line resulted in increased bone mass. It was found that caveolae and lipid microdomains are associated with mechanosensitive proteins on the osteoblast cell membrane, including P2X7R. Mechanosensitive response and signaling within bone tissue was modulated by endocytosis of activated receptors, and when caveolae were disrupted, researchers found increased calcium signaling and mineralization in response to ATP stimulation. This finding supports the hypothesis that disrupting endocytic pathways involving internalization of transmembrane proteins may be a novel method for enhancing the mechanosensitive response of bone. These studies have been performed at whole bone tissue level and with osteoblasts in vitro, however none have looked specifically at the main mechanosensory cells in bone, osteocytes. A recent study from our lab was the first instance of observing integrin cycling within osteocytes in vivo, and our current results support the conclusion that cholesterol- and caveolae-mediated endocytosis is an innate pathway to internalize and recycle integrin proteins within osteocytes. Our findings add to the consensus that caveolae-mediated endocytosis is a spatiotemporal controller of mechanosensitive proteins in osteocytes and may be an intriguing therapeutic target for modulating bone's response to mechanical stimulation. It is interesting that subcellular localization of C'Dots was not impacted while the uptake and clearance profiles were affected. Sex differences were not as visible in the MβCD clearance study as compared to the Dyngo4a results, however, taken together, a picture of sex differences in osteocyte C'Dot uptake and clearance emerges. Cholesterol is critical for both males and female receptor-mediated endocytosis while dynamin is only critical for males. This suggests that the canonical endocytic pathway of clathrin-mediated endocytosis does not play a large role in female osteocyte nanoparticle metabolism. Additionally, sex differences were observed in results between untargeted and integrin-targeted C'Dots. Male PEG C'Dot clearance retained significant differences from control with a MβCD injection while females did not, and neither PEG group reached significance with Dyngo4a application. Finally, subcellular localization results continued to identify sex differences in osteocyte endocytic pathways. Only males had a significant decrease in discrete subcellular localization of C'Dots under a drug condition compared to controls (FIG. 6). This may suggest that dynamin inhibition perturbs normal vesicular packaging of nanoparticles during uptake, but is only critical for that phenotype in males. Together, our results imply that endocytic pathways of nanoparticles in general and mechanosensitive transmembrane proteins like integrins in particular are unique between the sexes.

These results represent the first quantification of endocytosis and membrane dynamics in osteocytes in vivo. Our studies directly motivate future work to assess the impact of endocytic perturbation on osteocyte mechanosensitivity via the role of endocytic pathways on mechanosensitive protein availability. Additionally, the observed sex differences in endocytic pathway utilization for mechanosensitive proteins and normal nanoparticle metabolism in osteocytes is an intriguing and unexpected result that deserves further consideration within the bone community.

Observing the response of osteocytes to endocytic disruptors on mechanosensitive responses in vivo, including Ca²⁺ signaling and changes in gene expression during and after mechanical loading under drugged and undrugged conditions will be carried out. If localization and availability of mechanosensitive proteins can be controlled, pharmacological disruption of specific pathways is expected to be a way to modulate osteocyte reaction to mechanical stimulation.

### Example 3

This Example provides, inter alia, a description and characterization of inorganic nanoimaging agents and methods of using same.

Pharmacological perturbation of select endocytosis components was used to interrogate trafficking of fluorescent nanoparticles in osteocytes by visualizing uptake, subcellular localization, and clearance kinetics. Specifically, dynamin GTPase activity (implicated in clathrin endocytosis) and cholesterol-based lipid rafts (implicated in micropinocytosis) were targeted.

METHODS: Silica core poly(ethylene glycol) shell (core-shell) nanoparticles covalently encapsulating Cy5 dye (PE G-C'Dots) were created using a modified Stober condensation in water. RGD-C'Dots were created by surface functionalizing C'Dot surface with amino acid motifs that bind to the extracellular RGD domain of integrin proteins. Skeletally mature C57BL/6J mice age 16-18 weeks were used for these studies (n=4-5/group/sex). C'Dot Injections: Under inhaled isoflurane anesthesia, PEG- or RGD-C'Dots were injected subcutaneously above the third metatarsal (MT3) in the mouse hind paw at a concentration of 10 μM (FIG. 9). C'Dots were incubated for 45 minutes with mice allowed normal cage activity. Pre-incubation with Dyngo-4a (Abcam) or Methyl-β-cyclodextrin (Sigma Aldrich) for 30 minutes prior to C'Dot injection was used to inhibit dynamin activity or cholesterol lipid raft formation in treated groups. Imaging: After incubation, the MT3 was surgically isolated and stabilized using a metal 3-pin system in re-anesthetized mice'. The entire paw was submerged in a PBS bath and placed under a two-photon microscope (Bergamo II, ThorLabs). Osteocytes in the MT3 cortical bone were imaged with a 20× water immersion objective (XLUMPLFLN, Olympus), excitation wavelength of 1090 nm, and >647 nm long pass filter acquisition. Image Quantification: Mean intensity and cell count of the C'Dot tagged osteocytes were quantified in ImageJ (NIH) using 3D segmentation and were normalized to background intensity. Subcellular localization of C'Dots was quantified by eye from a single frame of the first z-stack of each clearance experiment. Cells were given a binary score for saturated or discretely localized. Clearance study z-stacks (35 μm depth) were taken every 15 minutes for 2.5 hours. Statistical analyses used GraphPad Prism software (2-Way ANOVA with multiple comparisons, p<0.05). All procedures were IACUC approved.

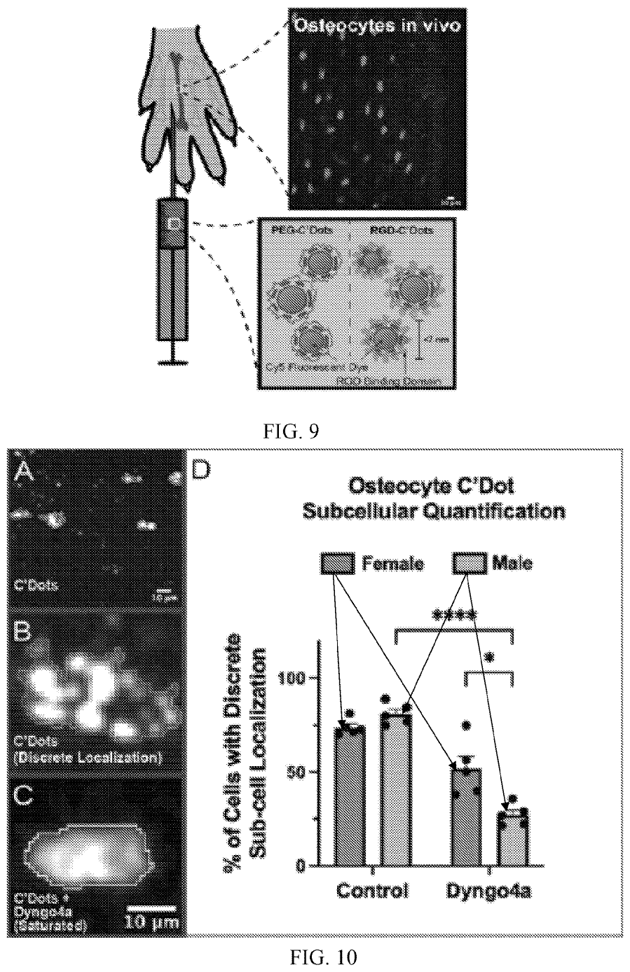

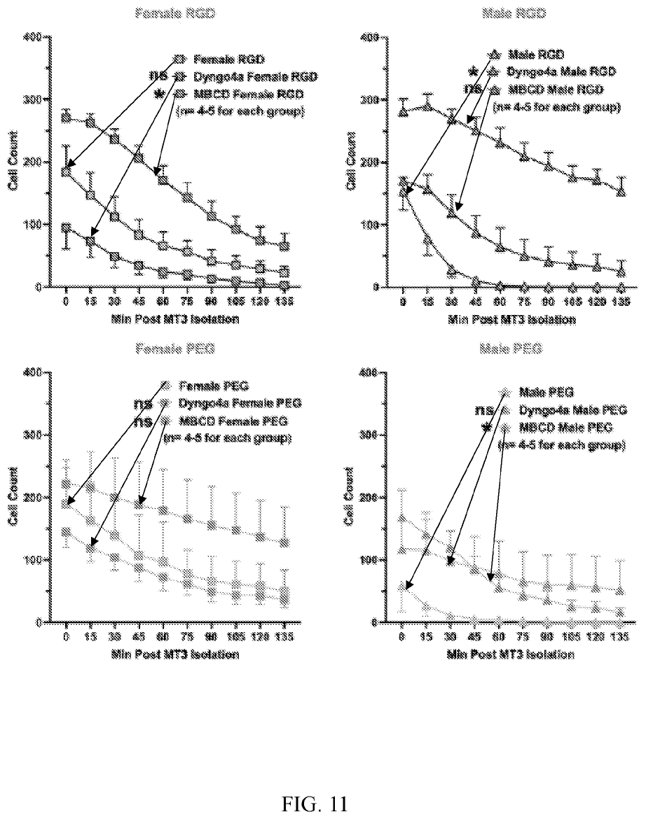

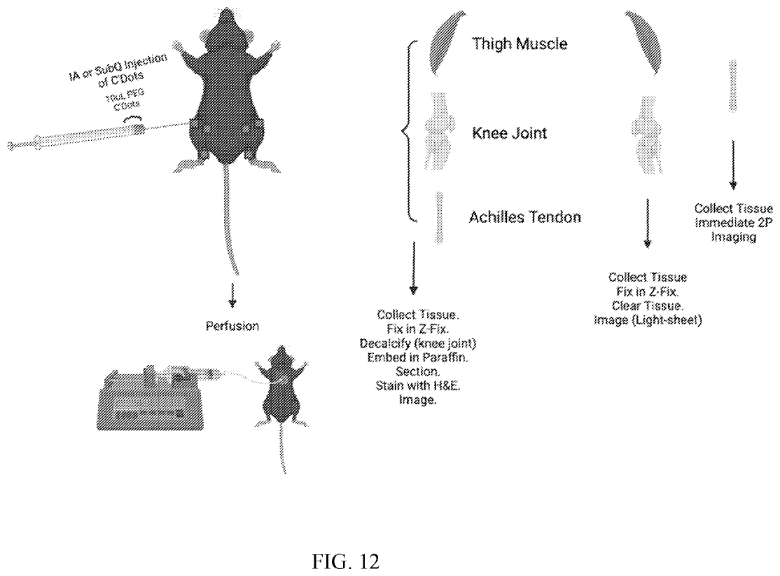

Results. Discrete subcellular localization of RGD C'Dot signal is seen in-75% of osteocyte cell bodies in untreated controls for both sexes (FIG. 10). Pre-incubation with dynamin inhibitor Dyngo4a reduced the number of cells with discrete localization to 25% in males (ANOVA, p<0.0001), and had no statistically significant impact in females. C' Dot clearance in untreated mice was sex-dependent (FIG. 11), with males losing signal much more rapidly than females'. Pre-incubation with Dyngo4a significantly increased RGD C'Dot signal intensity and retention in the male group (p<0.05, 2-Way ANOVA) with no effect in females. Pre-incubation with cholesterol-inhibitor M (3CD resulted in a significant increase i′ RGD C'Dot signal in the female group (p<0.05, 2-Way ANOVA) and a no effect in males. In both drug treatments, patterns in RGD C'Dot kinetics were replicated with PEG C'Dot clearance, but to a lesser extent and with more inter-animal variation.

Discussion Nanoparticle uptake and clearance can be modulated by small molecule endocytosis disruptors, as indicated by these novel intravital imaging studies. Dynamin is critical for rapid clearance and subcellular localization of RGD C'Dots in male mice, but appears to be less impactful for females. Cholesterol and lipid raft formation is necessary for normal RGD C'Dot clearance in female mice but is not significant in males. These data suggest that osteocyte endocytic pathways, specifically receptor mediated endocytosis of integrins, are differentially regulated between the sexes. Variability and reduced impact in PEG data supports that integrin targeting is distinct compared to non-targeted C'Dots. Together, these results imply that endocytosis of mechanosensitive transmembrane proteins like integrins is unique between the sexes. If localization and availability of mechanosensitive proteins can be controlled, pharmacological disruption of specific pathways may be a new way to modulate osteocyte reaction to mechanical stimulation.

These results represent the first quantification of endocytosis and membrane dynamics in osteocytes in vivo.

### Example 4

This Example provides, inter alia, a description and characterization of inorganic nanoimaging agents and methods of using same.

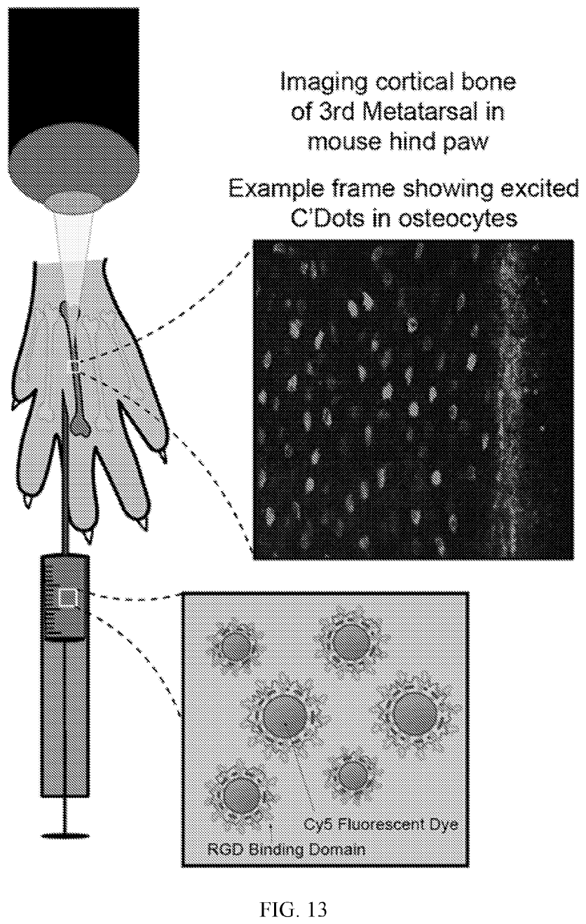

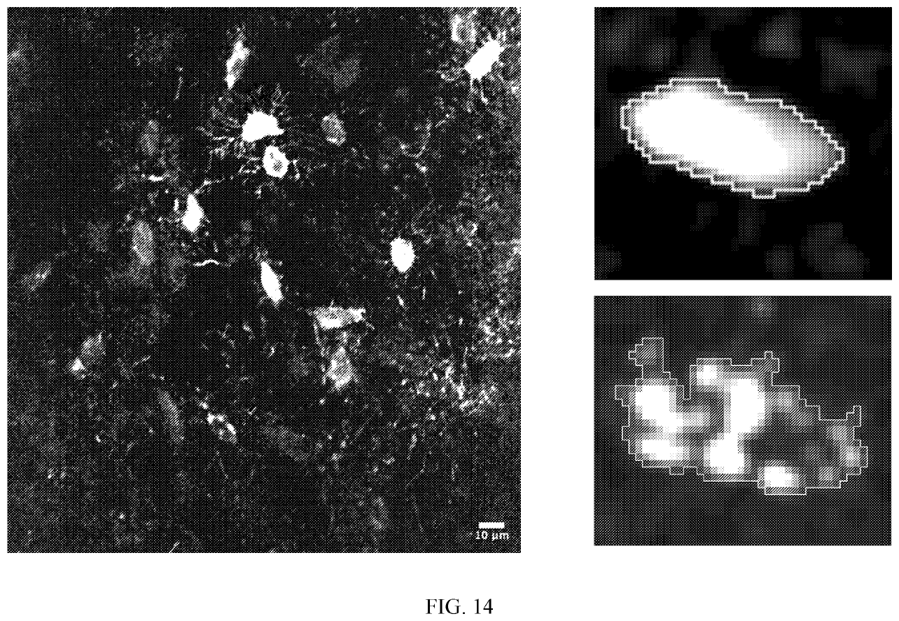

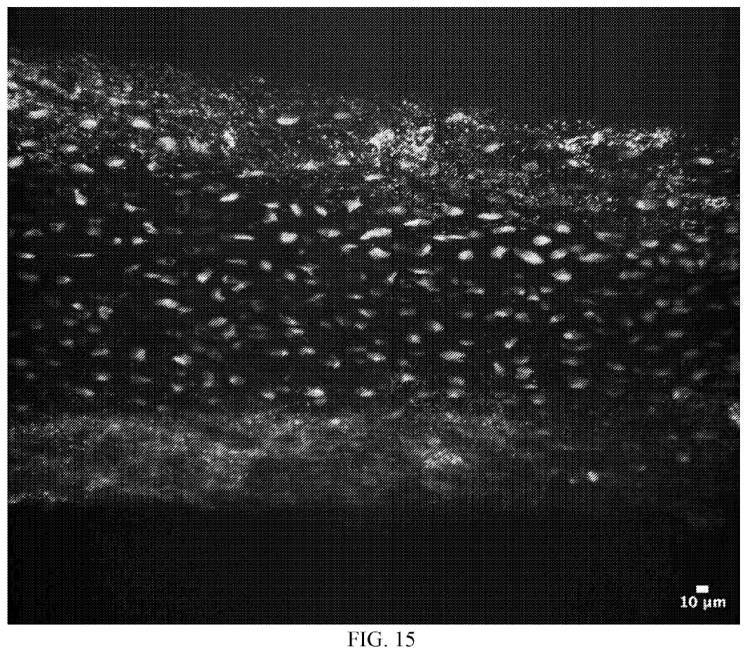

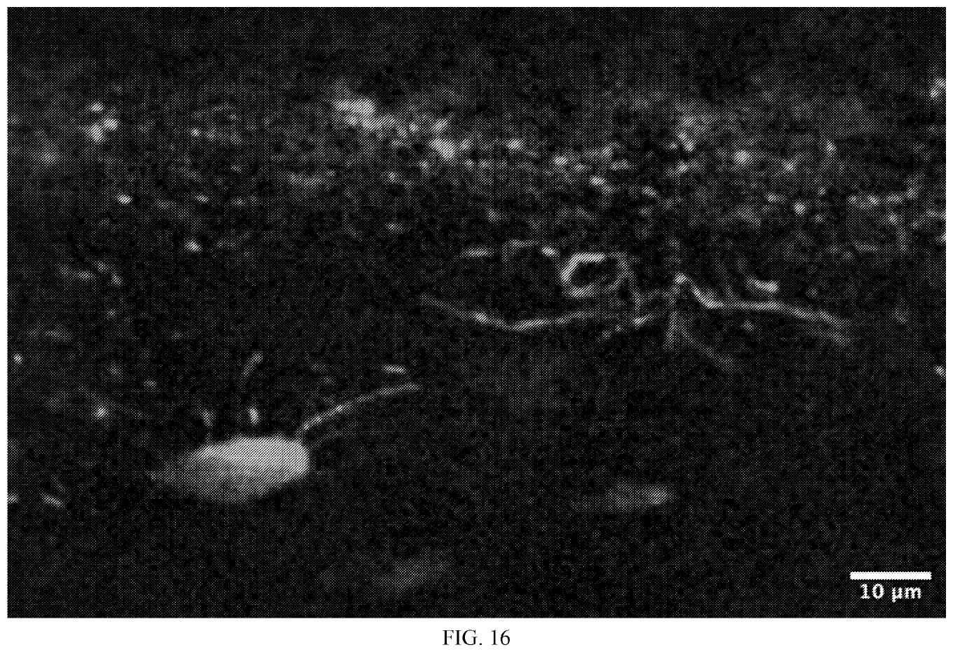

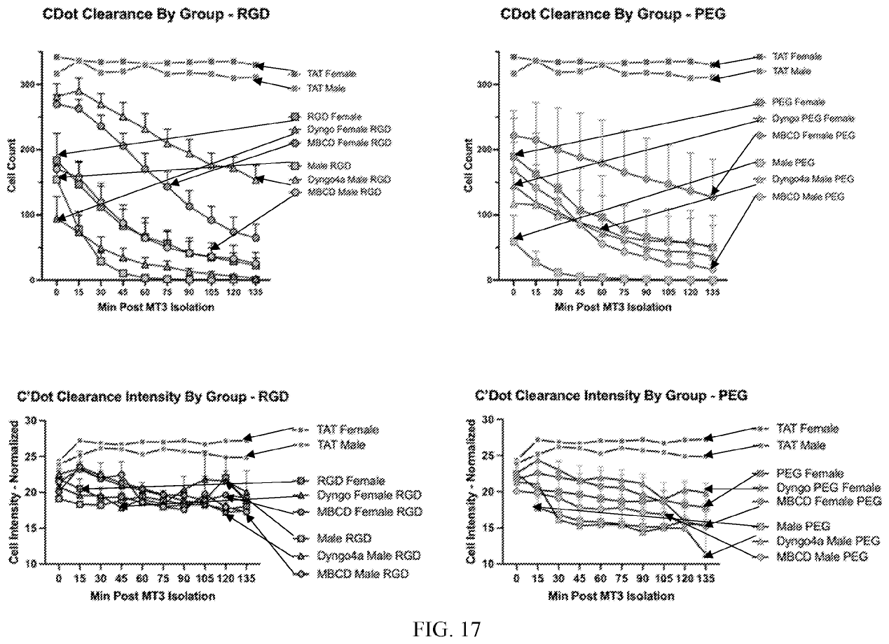

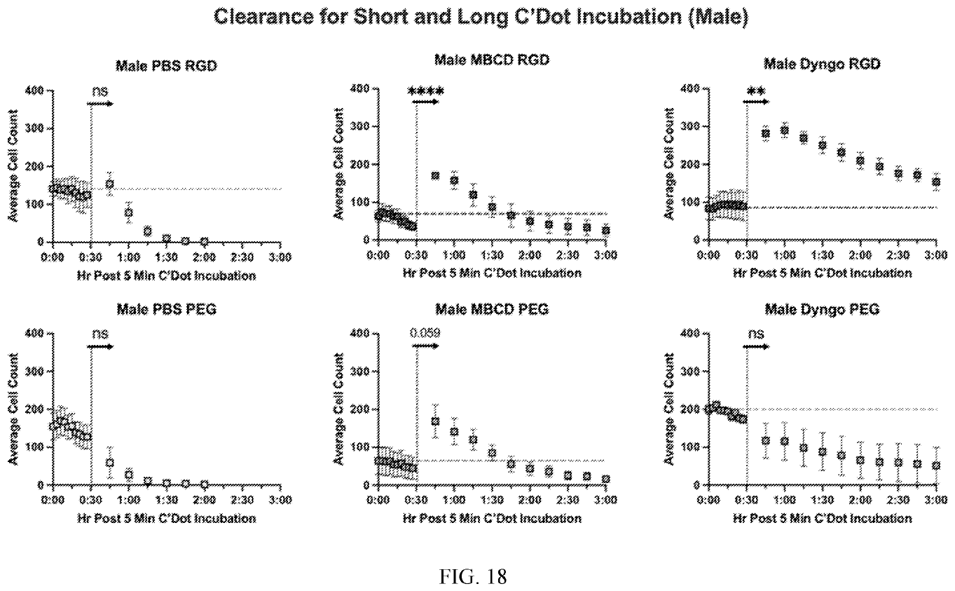

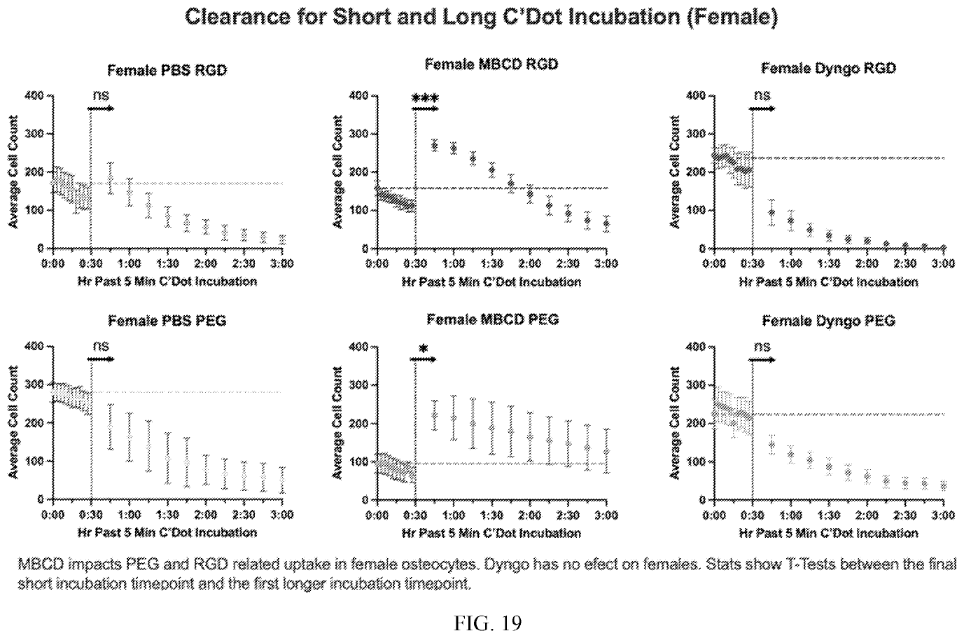

This example provides a description of the range of uses of C'Dots in musculoskeletal tissues and provides examples of subcellular targeting. The graphical abstracts and pilot experiments (see e.g., FIGS. 12-16) depict the range of C'Dot utility in musculoskeletal tissue, from targeting subcellular components like integrins or the actin cytoskeleton, to being able to observe subcellular activity like pH and endocytosis.

### Example 5

This Example provides, inter alia, a description and characterization of inorganic nanoimaging agents and methods of using same.

This example provides a description of inorganic nanoimaging agents for use in imaging methods and methods of using same.

### C'Dot Types:

PEG C'Dots—Pegylated C'Dots with Cy5 dye were used as an untargeted probe for visualizing osteocytes. They are taken up by the cells, but are not directed to bind to any specific cellular receptor. These C'Dots were used to study non-receptor mediated endocytosis within osteocytes in vivo. Size=<7 nm diameter

RGD C'Dots—Cyclic RGDyC C'Dots with Cy5 dye and RGD (integrin binding) peptides were used as an integrin targeted probe for studying and visualizing osteocytes. They are also taken up rapidly by cells, likely through receptor-mediated endocytosis of integrin and integrin-bound C'Dots. This endocytosis pathway would be differently regulated from non-receptor mediated endocytosis. Size=<7 nm diameter.

TAT C'Dots—Nanoparticles with TAT viral peptides and Cy5 dye were used as an endocytosis-avoiding control for studies in osteocytes in vivo. They are taken up into the cells, but retain signal and intensity over time for compared to both PEG and RGD C'Dots. The TAT peptides allow the nanoparticles to independently invade the cell cytoplasm, avoiding biologically regulated uptake mechanisms. Size=<7 nm diameter.

pH Sensing C'Dots—Experiments with pH sensing C'Dots, were carried out and have successfully localized them to osteocytes in vivo. We have observed both dyes used in the ratiometric analysis. This shows use of ratiometric C'Dots to measure cellular activity in vivo, from pH to ROS to calcium fluctuations. Size=<7 nm diameter.

Phalloidin Targeted C'Dots—Studies with phalloidin (actin cytoskeleton) targeting C'Dots were carried out. These Dots had Cy3 dye, and the signal within osteocytes was successfully visualized. A strong dendritic signal was observed with these C'Dots, supporting their targeting of the cytoskeleton. Size=<7 nm diameter.

aC'Dots—Aluminosilicate C'Dots (Cy5 dye) have application as a super-resolution imaging tool due to their unique blinking properties. Blinking was visualized a 2 photon microscope, and have seen osteocyte cellular uptake in vivo was observed.

C'Dot Rings and Cages—These types of C'Dots are expected to be useful in delivering a drug target to bone or other musculoskeletal cells.

### C'Dot Injection

All animal experiments were done in accordance with protocols approved by the Institutional Animal Care and Use Committee (IACUC) of Cornell University and observed NIH guidelines. Anesthetized wild-type C57BL6 mice from Jackson Labs were locally injected via Hamilton syringe with 10-15 μL of C'Dots (10 μM) subcutaneously above the 3^{rd} metatarsal in the hind paw (Male and female mice, 16-20 weeks old). The syringe was entered through the skin, into the subcutaneous space between the 2^{nd} and 3^{rd} metatarsals, near the distal joint. The depth of the needle into the space was ~0.5 cm, and the angle of insertion was ~10-20° as the C'Dots were injected. C'Dots are incubated in the foot prior to metatarsal isolation surgery for different amounts of time, from 45 seconds up to 96 hours. The majority of our experiments have been done with acute, 5 min incubations, or 45 minute incubations. In endocytosis experiments, different drugs are preincubated in the foot (injected in the same manner) prior to C'Dot application

### Metatarsal Isolation Surgery

After incubation of the injected C'Dots, isoflurane anesthetized mice (2-3% at 1 L/min) had the 3^{rd} metatarsal of their hind paw surgically isolated and stabilized prior to imaging. A vertical incision was made between the 2^{nd} and 3^{rd} metatarsals, from the distal joint to the ankle. A pick was used to remove anterior tendons and a metal pin was inserted beneath the distal end of the 3^{rd} metatarsal. The pin was situated in the middiaphysis of the bone and stabilized using a three pin loading method previously described (Lewis, 2017). The paw and isolated metatarsal were submerged in a DPBS bath to maintain a natural fluid environment prior to and throughout imaging.

### Imaging and Analysis

The structural marker potential of C'Dots for in vivo imaging of osteocytes was visualized with two-photon microscopy (TPM). Using a Ti:Sapphire laser (Coherent) with a 20× water immersion lens, imaging was performed with 1090-nm wavelength excitation and >647-nm bandpass filter acquisition for Cy5 dye based C'Dots. 920 nm was also used for pH sensing C'Dots, and 980 nm for Cy3 based C'Dots. The field-of-view for the system was set to 602×602 μm and a pixel count of 1024×1024. The isolated metatarsal and its osteocytes are imaged throughout a 35 μm z-stack (0.3 μm steps), starting ~20 μm beneath the surface of the bone. Other types of imaging could be performed also, from single snapshots, to video streaming. C'Dot fluorescence intensity measurements from individual osteocytes were taken by processing the z-stacks in ImageJ (NIH); these intensity values were normalized to background fluorescence in each image. Cells were selected based on thresholded signal and counted to find total number of cells with uptake in each imaging session.

### Main Experiments

Osteocyte C'Dot Clearance: How quickly the C'Dots were being taken up into osteocytes and how quickly they were degraded or recycled out of the cells was observed. Incubation time was varied from 45 seconds out to 96 hours. Signal was taken up almost immediately, with the 45 second incubation providing strong cellular signal. Most C'Dot signal was lost after a 4 hour incubation. To observe how C'Dot signal changed over time in the cells, a mid-incubation of 45 min was used and then the C'Dots imaged every 15 minutes for 2.5 hours. This allowed us to create a kinetic curve of decreasing C'Dot signal over time. Surprisingly, these curves were different between the sexes but not between PEG and RGD C'Dots.

Osteocyte C'Dot Endocytosis and Metabolism: C'Dots and their rapid uptake and clearance from osteocytes was used to study endocytosis in living bone tissue. In endocytosis experiments, different drugs are preincubated in the foot (injected in the same manner) prior to C'Dot application and incubation. Drugs to target different types of endocytic pathways, including non-receptor mediated endocytosis and clathrin mediated endocytosis, were used. The same clearance imaging was performed to see how the drugs impact the cellular uptake and retention of the nanoparticles over time.

Additionally, this experiment was performed again with a short, 5 minute incubation and more rapid imaging to see what the acute impacts of the drugs were on C'Dot uptake.

Osteocyte Morphology: A 5 minute incubation time and a smaller depth of imaging was used to observe and quantify osteocyte dendritic morphology.

Osteocyte Subcellular Localization: In all of these experiments, changes in subcellular localization of C'Dots were observed, providing support for the use of C'Dots to target and visualizing subcellular compartments like endosomes.

### Representative Data

TAT C'Dots showed distinctly higher uptake and retention of signal compared to both RGD and PEG C'Dots over time. This supports their use as an endocytic control and their ability to circumvent endocytic degradation/recycling mechanisms.

Although the present disclosure has been described with respect to one or more particular embodiments and/or examples, it will be understood that other embodiments and/or examples of the present disclosure may be made without departing from the scope of the present disclosure.

1. A method of imaging of one or more bone(s) or any portion thereof, one or more musculoskeletal tissue(s) or any portion thereof, or any combination thereof in a sample or in an individual or a portion thereof, the method comprising:
contacting the sample or the individual with a plurality of one or more inorganic nanoimaging agent(s), wherein each inorganic nanoimaging agent(s) of the plurality of inorganic nanoimaging agent(s) independently comprises one or more dye group(s), one or more sulfur atom group(s), one or more radioisotope group(s), one or more heavy atom group(s), one or more iodide group(s), or any combination thereof, wherein the dye group(s), the sulfur atom group(s), the radioisotope group(s), the heavy atom group(s), the iodide(s), or any combination thereof, if present, are independently disposed on a surface of an inorganic nanoimaging agent, covalently or non-covalently bonded to a matrix of the inorganic nanoimaging agent;
directing excitation electromagnetic radiation into the sample or the individual, thereby exciting at least one of the one or more dye group(s), if present, one or more sulfur atom group(s), if present, one or more radioisotope group(s), if present, one or more heavy atom group(s), if present, one or more iodide group(s), if present, or the combination thereof resulting in excitation and emission of electromagnetic radiation from the one or more dye group(s), if present, one or more sulfur atom group(s), if present, one or more radioisotope group(s), if present, one or more heavy atom group(s), if present, one or more iodide group(s), if present, or the combination thereof;
detecting at least a portion of the emitted electromagnetic radiation, the detected electromagnetic radiation having been emitted by the one or more dye group(s), if present, one or more sulfur atom group(s), if present, one or more radioisotope group(s), if present, one or more heavy atom group(s), if present, one or more iodide group(s), if present, or the combination thereof; and
processing signals corresponding to the detected electromagnetic radiation to provide one or more image(s) of the bone(s) or the portion thereof, the musculoskeletal tissue(s) or the portion thereof, or the combination thereof in the sample, the individual, or the portion thereof.

2. A method of claim 1, wherein the inorganic nanoimaging agent(s) is/are chosen from silica nanoimaging agents, aluminosilicate nanoimaging agents, or the like, or any combination thereof.

3. A method of claim 1, wherein the inorganic nanoimaging agent(s) is/are chosen from nanoparticles, nanorings, nanocages, or any combination thereof.

4. A method of claim 3, wherein the inorganic nanoimaging agent(s) further comprise(s) a plurality of polyethylene glycol (PEG) groups disposed on at least a portion of a surface or all of the surfaces of the inorganic nanoimaging agent nanoparticle.

5. A method of claim 1, wherein the inorganic nanoimaging agent(s) comprise a longest linear dimension of about 2 nm to about 1 micron.

6. A method of claim 1, wherein at least a portion of the dye groups(s) is/are organic dye group(s) or a combination of organic dye group(s).

7. A method of claim 6, wherein at least a portion the dye group(s) is/are fluorescent dye groups(s), fluorescent protein groups(s), or any combination thereof.

8. A method of claim 6, wherein the organic dye group(s) is/are chosen from cyanine dyes, rhodamine dyes, coumarin dyes, boron-dipyrromethene (BODIPY) dyes, xanthene dyes, eosin dyes, carbopyronine dyes, methylene blue, fluorescein, Acridine Orange, structural derivatives thereof, any groups derived therefrom, and any combination thereof.

9. A method of claim 1, wherein the heavy atom group(s) is/are chosen from iodine atom, bromine atom, metal ions, structural derivatives thereof, any group derived therefrom, and any combination thereof.

10. A method of claim 9, wherein the metal ions are Au ions, Ag ions, Pb ions, Ti ions, Bi ions, Pt ions, In ions, Sn ions, Sb ions or Pd ions.

11. A method of claim 1, wherein the contacting is administering an effective amount of one or more composition(s) comprising the inorganic nanoimaging agent(s) to the individual.

12. A method of claim 11, wherein the administering is subcutaneous administration, intravenous administration, intraarticular administration, or intramarrow administration.

13. A method of claim 1, wherein the electromagnetic radiation is directed into the individual.

14. A method of claim 13, wherein the electromagnetic radiation is directed into a region, wherein the region is within the individual.

15. A method according to claim 1, wherein the electromagnetic radiation comprises one or more wavelengths at about 400 to about 1700 nm.

16. A method of claim 1, wherein the electromagnetic radiation is provided by one or more laser(s).

17. A method of claim 16, wherein the electromagnetic radiation is provided by a tunable wavelength laser.

18. A method of claim 1, wherein the directing, detecting, and processing is fluorescence imaging, bioluminescence imaging, or any combination thereof.

19. A method of claim 1, wherein at least a portion of the inorganic nanoimaging agent(s) is/are aluminosilicate nanoimaging agent(s) and the imaging is super-resolution imaging.

20. A method of claim 19, wherein the image(s) are fluorescence image(s) or a sequence of fluorescence image(s) which can be processed to obtain a super-resolution optical image of the sample, the individual, or the portion thereof.

21. A method of claim 1, wherein the method is an optical super-resolution microcopy method chosen from ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM).

22. A method of claim 1, the method further comprising targeting, diagnosing, treating, preventing, or any combination thereof, a current or potential disease, disease state, condition, disorder, side effect, or any combination thereof, in an individual.

23. A method of claim 22, wherein the sample is a biopsy sample or a resected tissue sample.

24. A method of claim 22, wherein the current or potential disease, disease state, condition, disorder, side effect, or any combination thereof, is chosen from infections, cancers, inflammatory conditions, and any combination thereof.

25. A method of claim 22, wherein at least a portion of the inorganic nanoimaging agent(s) independently comprises one or more drug group(s), one or more radioisotope group(s), or any combination thereof, and the contacting results in treatment of the individual.

26. A method of claim 1, wherein at least a portion of the inorganic nanoimaging agent(s) independently comprises one or more targeting group(s).

27. A method of claim 26, wherein the targeting group(s) are chosen from integrins, transmembrane proteins, intracellular signaling proteins, and any combination thereof.

28. A method of claim 24, wherein the current or potential disease, disease state, condition, disorder, side effect, or any combination thereof is cancer, and the method further comprises one or more chemotherapy treatment(s), one or more radiation treatment(s), one or more photodynamic therapy treatment(s), one or more surgical intervention(s) or any combination thereof.

29. A method of determining a presence, an absence, or a concentration of an analyte proximate to or within one or more bone(s) or any portion thereof, one or more musculoskeletal tissue(s) or any portion thereof, or any combination thereof in a sample, an individual, or a portion thereof using one or more inorganic nanoimaging agent(s) comprising:
contacting the sample or individual with one or more inorganic nanoimaging agent(s) each inorganic nanoimaging agent comprising: one or more reference dye groups(s), where each reference dye group is covalently bound to and encapsulated in a network of the inorganic nanoimaging agent, and/or one or more sensing dye groups(s), wherein the sensing groups are capable of interacting with the sample, the individual, or the portion thereof, a plurality of polyethylene glycol (PEG) groups disposed on at least a portion of a surface of the inorganic nanoimaging agent;
optionally, incubating an inorganic nanoimaging agent(s) with the sample or in the individual; determining a presence, an absence, or a concentration of the analyte in an individual region of a first object plane using ratiometric sensing;
localizing with resolution below Abbe's diffraction limit at least a portion of the individual inorganic nanoimaging agent(s);
determining the presence, the absence, or a concentration of the analyte at a position of one of the one or more of the inorganic nanoimaging agent(s) using the presence, the absence, or the concentration of the analyte obtained using the ratiometric sensing and the localization of the inorganic nanoimaging agent(s); and
optionally, averaging a fluorescence intensity ratio of a desired number of individual inorganic nanoimaging agent(s) in proximity to an individual inorganic nanoimaging agent to assign an average fluorescence intensity ratio to one or more of the individual inorganic nanoimaging agent(s), wherein an average fluorescence intensity ratio assigned to the individual inorganic nanoimaging agent(s) corresponds to the presence, the absence, or the concentration of the analyte in the sample, the individual, or any portion thereof.

30. A method of claim 29, wherein the localizing with resolution below Abbe's diffraction limit at least a portion of the individual inorganic nanoimaging agent(s) is in a second object plane, wherein the second object plane comprises at least a portion of the first object plane.

31. A method of claim 29, wherein the determining the presence, the absence, or the concentration of the analyte in the individual region of the first object plane using ratiometric sensing and the localizing with resolution below Abbe's diffraction limit at least a portion of or all of the individual inorganic nanoimaging agent(s) are each carried out using optical super-resolution microscopy (OSRM) imaging.

32. A method of claim 29, wherein the presence, the absence, or the concentration of the analyte in the sample, the individual, or the portion thereof is determined at a position of one or more of the inorganic nanoimaging agent(s).

33. A method of claim 29, wherein the analyte is chosen from hydrogen ions, oxidants, antioxidants, oxygen, reactive oxygen species (ROS), nitric oxide, chloride ions, metals, metal ions, and any combination thereof.

34. A method of claim 29, wherein the analyte is hydrogen ions and a local pH is determined at a position of at least a portion of the inorganic nanoimaging agent(s) in the sample, the individual, or the portion thereof.

35. A method of claim 29, wherein the individual sensing dye group(s) is/are capable of sensing pH, sensing redox status, sensing the presence or absence of oxygen, sensing the presence or absence of reactive oxygen species (ROS), sensing the presence or absence of chloride ions, sensing the presence or absence of nitric oxide, and/or sensing the presence or absence of one or more metal(s) and/or metal ion(s).

36. A method of claim 29, wherein at least a portion of the inorganic nanoimaging agent(s) further comprise one or more targeting group(s), one or more therapeutic group(s), one or more diagnostic group(s), or any combination thereof.

37. A method of claim 29, wherein the method comprises an OSRM method chosen from ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM).

38. A method of claim 29, wherein the inorganic nanoimaging agent(s) individually have at least one linear dimension of about 1 to about 30 nm.

39. A method of claim 29, wherein the individual is a human or a non-human animal.

40. A composition for imaging one or more bone(s) or any portion thereof, one or more musculoskeletal tissue(s) or any portion thereof, or any combination thereof in a sample or in an individual, comprising:
a plurality of one or more inorganic nanoimaging agent(s), wherein each inorganic nanoimaging agent(s) of the plurality of inorganic nanoimaging agent(s) independently comprises one or more dye group(s), one or more sulfur atom group(s), one or more radioisotope group(s), one or more heavy atom group(s), one or more iodide group(s), one or more targeting group(s), or any combination thereof, wherein the dye group(s), the sulfur atom group(s), the radioisotope group(s), the heavy atom group(s), the iodide group(s), the targeting group(s), or the combination(s) thereof, if present, are independently disposed on a surface of an inorganic nanoimaging agent, covalently or non-covalently bonded to a matrix of the inorganic nanoimaging agent.

41. A composition of claim 40, wherein the dye group(s) is/are chosen from cyanine dyes, rhodamine dyes, coumarin dyes, boron-dipyrromethene (BODIPY) dyes, xanthene dyes, eosin dyes, carbopyronine dyes, methylene blue, fluorescein, Acridine Orange, structural derivatives thereof, any groups derived therefrom, and any combination thereof.

42. A composition of claim 40, further comprising one or more pharmaceutically acceptable excipient(s).

43. A composition of claim 40, further comprising a concentration of the inorganic nanoimaging agent(s) of about 10 nanomolar to about 100 micromolar.

44. A composition of claim 40, wherein at least a portion of the inorganic nanoimaging agent(s) independently comprises one or more drug group(s), one or more radioisotope group(s), or any combination thereof.

45. A composition of claim 40, wherein the composition is suitable for subcutaneous administration, intravenous administration, intraarticular administration, or intramarrow administration.

46. An inorganic nanoimaging agent comprising one or more targeting group(s) independently bonded to a matrix of the inorganic nanoimaging agent and/or one or more dye group(s) independently bonded to the matrix of the inorganic nanoimaging agent.

47. An inorganic nanoimaging agent of claim 46, wherein the matrix is a silicate or aluminosilicate matrix.

48. An inorganic nanoimaging agent of claim 46, further comprising a plurality of polyethylene glycol (PEG) groups disposed on at least a portion of a surface or all of the surfaces of the inorganic nanoimaging agent(s).

49. An inorganic nanoimaging agent of claim 46, wherein the inorganic nanoimaging agent comprises a diameter of about 1 nm to about 8 nm.

50. An inorganic nanoimaging agent of claim 46, wherein the inorganic nanoimaging agent comprises a diameter of about 7 nm or less.

51. An inorganic nanoimaging agent of claim 46, wherein the one or more dye group(s) are independently chosen from RhG, TMR, Cy3, Cy5, Cy5.5, DY782, and CW800.

52. An inorganic nanoimaging agent of claim 46, wherein the one or more targeting group(s) are independently chosen from integrin binding peptide(s), TAT viral peptide(s), and phalloidin targeting group(s).

53. A kit comprising one or more inorganic nanoimaging agent(s) and/or one or more composition(s) comprising the inorganic nanoimaging agent(s), and instructions for use of the inorganic nanoimaging agent(s) and/or the composition(s).

54. A kit of claim 53, wherein the inorganic nanoimaging agent(s) are chosen from:
aluminosilicate core-organic ligand shell nanoparticles, each of the aluminosilicate core-organic ligand shell nanoparticles comprising:
an aluminosilicate core,
one or more reference dye group(s) covalently bound to and encapsulated in the aluminosilicate network of the aluminosilicate core-organic ligand shell nanoparticle,
one or more sensing dye group(s) capable of analyte sensing covalently bound to the aluminosilicate core network, wherein the one or more reference dye group(s) and the one or more sensing dye group(s) do not interfere with each other and/or one or more sensing dye group(s) capable of analyte sensing is/are disposed on at least a portion of a surface of the aluminosilicate core, and
a plurality of PEG groups disposed on at least a portion of a surface of the aluminosilicate core;
aluminosilicate core-aluminosilicate shell-organic shell nanoparticles, each of the aluminosilicate core-aluminosilicate shell-organic shell nanoparticles comprising:
an aluminosilicate core,
one or more reference dye group(s) covalently bound to and encapsulated in the aluminosilicate network of the aluminosilicate core,
an aluminosilicate shell disposed on at least a portion of a surface of the aluminosilicate core,
one or more sensing dye group(s) capable of analyte sensing covalently bound to and encapsulated in the aluminosilicate network of the aluminosilicate shell,
optionally, one or more sensing dye group(s) capable of analyte sensing disposed on at least a portion of a surface of the aluminosilicate shell, and
a plurality of PEG groups disposed on at least a portion of a surface of the aluminosilicate shell; and
any combination thereof.

55. A kit of claim 53, wherein the inorganic nanoimaging agent(s) individually have at least one linear dimension of about 1 nm to about 30 nm.
